(19) United States
(12) Patent Application Publication
(10) Pub. No.: US 2023/0033102 A1
(43) Pub. Date: Feb. 2, 2023

(54) MONETIZATION OF ANIMAL DATA

(71) Applicant: SPORTS DATA LABS, INC., Royal Oak, MI (US)

(72) Inventors: Mark GORSKI, Royal Oak, MI (US); Vivek KHARE, Cupertino, CA (US); Stanley MIMOTO, Bethel Island, CA (US)

(73) Assignee: SPORTS DATA LABS, INC., Royal Oak, MI (US)

(21) Appl. No.: 16/977,454

(22) PCT Filed: Apr. 15, 2020
(86) PCT No.: PCT/US2020/028355

§ 371 Date: Sep. 1, 2020

Related U.S. Application Data
(60) Provisional application No. 62/834,131, filed on Apr. 15, 2019.
(60) Provisional application No. 62/912,210, filed on Oct. 8, 2019.

(51) Int. Cl. G06F 16/2457; G06F 16/23

(52) U.S. Cl. CPC G06F 16/2457; G06F 16/2365

(57) ABSTRACT

A system for monetizing animal data includes a source of animal data that can be transmitted electronically. Characteristically, the source of animal data includes at least one sensor. An intermediary server receives and collects the animal data such that collected data has attached thereto metadata. The metadata includes at least one of the origination of the animal data or personal attributes of individuals from which the animal data originated. The intermediary server provides requested animal data to a data acquirer for consideration. The requested animal data may include simulated animal data. The intermediary server will also distribute at least a portion of the consideration to at least one stakeholder. The intermediary server includes a single computer server or a plurality of interacting computer servers.

### CROSS-REFERENCE TO RELATED APPLICATIONS

This application claims the benefit of U.S. Provisional Application Serial No. 62/834,131 filed Apr. 15, 2019 and U.S. Provisional Application Serial No. 62/912,210 filed Oct. 8, 2019, the disclosures of which are hereby incorporated in their entirety by reference herein.

### TECHNICAL FIELD

In at least one aspect, the present invention is related to systems for monetizing animal data.

### BACKGROUND

The continuing advances in the availability of information over the Internet have substantially changed the way that business is conducted. Simultaneous with this information explosion, sensor technology, and in particular, biosensor technology has also progressed. In particular, miniature biosensors that measure electrocardiogram signals, blood flow, body temperature, perspiration levels, or breathing rate are now available. However, centralized service providers that collect and organize information collected from such biosensors for the purposes of monetizing such information do not exist.

Accordingly, there is a need for systems that collect, organize and classify sensor data from an individual or group of individuals to make such data available for sale.

### SUMMARY

In at least one aspect, a system for monetizing animal data is provided. The system includes a source of animal data that includes at least one sensor. The animal data can be transmitted electronically. Characteristically, the source of animal data includes at least one sensor. An intermediary server receives and collects the animal data such that collected data has metadata attached thereto. The metadata includes at least one of origination of the animal data or one or more personal attributes of the one or more individuals from which the animal data originated. The intermediary server provides requested animal data to a data acquirer for consideration. The intermediary server also distributes at least a portion of the consideration to at least one stakeholder. The intermediary server includes a single computer server or a plurality of interacting computer servers.

In another aspect, a system for monetizing animal data is provided. The system includes a source of animal data that can be transmitted electronically, which includes at least one sensor. An intermediary server receives and collects the animal data. The intermediary server also provides requested animal data to a data acquirer for consideration. Characteristically. at least a portion of the requested or provided animal data is simulated animal data. The intermediary server distributes at least a portion of the consideration to at least one stakeholder. The intermediary server includes a single computer server or a plurality of interacting computer servers.

In another aspect, the animal data used in the system for monetizing animal data is human data.

In another aspect, the system for monetizing animal data can provide another dimension for one or more users to interact with athletic events. In particular, the present invention may provide a new dimension to sports wagering, including events involving humans or other mammals (e.g., horse racing).

In still another aspect, the system for monetizing animal data can provide purchasers of data (e.g., individuals, pharmaceutical companies, insurance companies, healthcare companies, military organizations, research institutions) an ability to acquire animal data for its particular use cases via an eCommerce website or platform such as a data marketplace.

### BRIEF DESCRIPTION OF THE DRAWINGS

FIG. 1 provides a schematic illustration of a system that monetizes and collects animal data.

FIG. 2 provides an illustration of a window through which a user can interact with an embodiment of the monetization system of FIG. 1.

FIG. 3A provides an illustration of a window presented to a data provider.

FIG. 3B provides an illustration of a window listing tags determined from the selection made in FIG. 3A.

FIG. 4 provides an illustration of a window showing sensor information.

FIG. 5 provides an illustration of a window showing active sensors and associated data that has been collected by sensors. The illustration also shows other data uploaded and the user’s ability to set a price for any data type from any selected sensor or uploaded data.

FIG. 6 provides an illustration of a window providing additional detail related to any given collected data set, as well as providing additional functionality to a user.

FIG. 7 provides an illustration of a summary window that displays the fees collected for any individual data provider.

FIG. 8 provides an illustration of a window that illustrates the scenario when a data acquirer requests non-live data.

FIG. 9 provides an illustration of an acquisition window (e.g., purchase window) that is displayed aftcr a data acquirer has found and selected the one or more data sets from the one or more individuals the data acquirer is interested in acquiring.

FIG. 10 provides an illustration of a window that includes a section in which a data acquirer can set a price for data sets and acquire additional data and data-related offerings.

FIG. 11 provides an illustration of a window display when one or more requested data sets are not available.

FIG. 12 provides an illustration of a window presented when requested data sets are not available, as well as functionality that enables the acquirer to set the price for requested data.

FIG. 13 provides an illustration of a window presented to a data provider that presents an opportunity to create data to the exact specifications of the data acquirer in exchange for consideration.

FIG. 14 provides an illustration of a window that illustrates the scenario when a data acquirer requests live data.

FIG. 15 provides an illustration of a window showing rights options associated with a potential purchase.

FIG. 16 provides an illustration of a window that illustrates an example of how revenue may be dispersed from a transaction.

FIG. 17 provides an illustration of a window that illustrates an example of how revenue can be allocated or adjusted, as well as the addition or removal of one or more stakeholders related to a transaction.

FIG. 18 provides a flowchart illustrating a user interacting with a third-party publisher site having an advertisement that utilizes animal data sets and in particular, human data sets.

FIG. 19 provides an illustration of a video game whereby user can purchase simulated data based in part on real animal data to provide a user with one or more advantages within the game.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

When a computing device is described as performing an action or method step, it is understood that the computing device is operable to perform the action or method step typically by executing one or more lines of source code. The actions or method steps can be encoded onto non-transitory memory (e.g., hard drives, optical drive, flash drives, and the like).

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” and “multiple” as a subset.

Throughout this application, where publications are referenced, the disclosures of these publications in their entireties are hereby incorporated by reference into this application to more fully describe the state of the art to which this invention pertains.

The term “server” refers to any computer or computing device (including, but not limited to, desktop computer, notebook computer, laptop computer, mainframe, mobile phone, smart watches/glasses, AR/VR headset, and the like), distributed system, blade, gateway, switch, processing device, or combination thereof adapted to perform the methods and functions set forth herein.

The term “computing device” refers generally to any device that can perform at least one function, including communicating with another computing device. In a refinement, a computing device includes a central processing unit that can execute program steps and memory for storing data and a program code. As used herein, a computing subsystem is a computing device.

The processes, methods, or algorithms disclosed herein can be deliverable to/implemented by a computing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, other magnetic and optical media, and shared or dedicated cloud computing resources. The processes, methods, or algorithms can also be implemented in a software executable object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

The terms “subject” or “individual” are synonymous and refer to a human or other animal, including birds and fish, as well as all mammals including primates (particularly higher primates), horses, sheep, dogs, rodents, guinea pigs, cats, whales, rabbits, and cows. The one or more subjects may be, for example, humans participating in athletic training or competition, horses racing on a track, humans playing a video game, humans monitoring their personal health, humans providing their data to a third party, humans participating in a research or clinical study, or humans participating in a fitness class. A subject or individual can also be a derivative of a human or other animal (e.g., lab-generated organism derived at least in part from a human or other animal), one or more individual components, elements, or processes of a human or other animal that comprise the human or other animal (e.g., cells, proteins, biological fluids, amino acid sequences, tissues, hairs, limbs), or one or more artificial creations that share one or more characteristics with a human or other animal (e.g., lab-grown human brain cells that produce an electrical signal similar to that of human brain cells). In a refinement, the subject or individual can be a machine (e.g., robot, autonomous vehicle, mechanical arm) or network of machines programmable by one or more computing devices that share at least one biological function with a human or other animal and from which one or more types of biological data can be derived, which may be, at least in part, artificial in nature (e.g., data from artificial intelligence-derived activity that mimics biological brain activity).

The term “animal data” refers to any data obtainable from, or generated directly or indirectly by, a subject that can be transformed into a form that can be transmitted (e.g., wireless or wired transmission) to a server or other computing device. Animal data includes any data that can be obtained from one or more sensors or sensing equipment/systems, and in particular, biological sensors (biosensors). Animal data can also include descriptive data, auditory data, visually-captured data, neurologically-generated data (e.g., brain signals from neurons), data that can be manually entered related to a subject (e.g., medical history, social habits, feelings of a subject), and data that includes at least a portion of animal data. In a refinement, the term “animal data” is inclusive of any derivative of animal data. In another refinement, animal data includes at least a portion of simulated data. In yet another refinement, animal data is inclusive of simulated data.

The term “artificial data” refers to artificially-created data that is derived from or generated using, at least in part, real animal data or its one or more derivatives. It can be created by running one or more simulations utilizing one or more artificial intelligence techniques or statistical models, and can include one or more signals or readings from one or more non-animal data sources as one or more inputs. Artificial data also includes any artificially-created data that shares at least one biological function with a human or other animal (e.g., artificially-created vision data, artificially-created movement data). It is inclusive of “synthetic data,” which can be any production data applicable to a given situation that is not obtained by direct measurement. Synthetic data can be created by statistically modeling original data and then using those models to generate new data values that reproduce at least one of the original data’s statistical properties. For the purposes of the presently disclosed and claimed subject matter, the terms “simulated data” and “synthetic data” are synonymous and used interchangeably with “artificial data,” and reference to any one of the terms should not be interpreted as limiting but rather as encompassing all possible meanings of all the terms.

The term “insight” refers to one or more descriptions that can be assigned to a targeted individual that describe a condition or status of the targeted individual. Examples include descriptions of stress levels (e.g., high stress, low stress), energy levels, fatigue levels, and the like. Insights may be quantified by one or more numbers, or a plurality of numbers, and may be represented as a probability or similar odds-based indicator. Insights may also be characterized by one or more other metrics, readings, insights, graphs, charts, plots, or indices of performance that are predetermined (e.g., visually such as a color or physically such as a vibration).

### Abbreviations

“AFE” means analog front end.

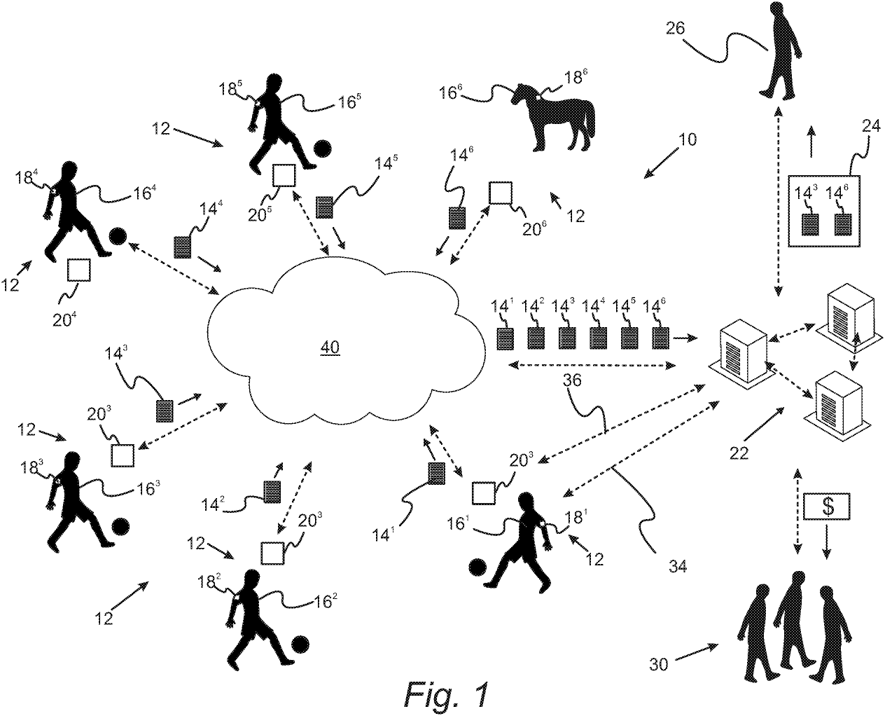

With reference to FIG. 1, a schematic of a system for monetizing animal data is provided. Monetization system 10 includes a source 12 of animal data 14ⁱ that can be transmitted electronically. Characteristically, source 12 of animal data includes at least one sensor 18ⁱ. Targeted individual 16ⁱ is the subject from which corresponding animal data 14ⁱ is collected. Label i is merely an integer label from 1 to iₘₐₓ associated with each targeted individual where iₘₐₓ is the total number of individuals, which can be 1 to several thousand or more. In this context, animal data refers to data related to a subject’s body derived, at least in part, from one or more sensors and, in particular, biological sensors (biosensors). In many useful applications, the subject is a human (e.g., an athlete), and the animal data is human data.

Biological sensors (biosensors) collect biosignals which in the context of the present embodiment are any signals or properties in, or derived from, subjects that can be continually or intermittently measured, monitored, observed, calculated, computed, inputted, or interpreted, including both electrical and non-clectrical signals, measurements, and artificially-generated information. A biological sensor can gather biological data such as physiological, biometric, chemical, biomechanical, genetic, genomic, location, or other biological data from one or more targeted individuals. For example, some biosensors may measure, or provide information that can be converted into or derived from, biological data such as eye-tracking data (e.g., pupillary response, movement, EOG-related data), blood flow/volume data (e.g., PPG data, pulse transit time, pulse arrival time), biological fluid data (e.g., analysis derived from blood, urine, saliva, sweat, cerebrospinal fluid), body composition data (e.g., BMI, % body fat, protein/muscle), biochemical composition data, biochemical structure data, pulse data, oxygenation data (e.g., SpO2), core body temperature data, skin temperature data, galvanic skin response data, perspiration data (e.g., rate, composition), blood pressure data (e.g., systolic, diastolic, MAP), hydration data (e.g., fluid balance I/O), heart-based data (e.g., heart rate, average HR, HR range, heart rate variability, HRV time domain, HRV frequency domain, autonomic tone, BCG-related data including PR, QRS, QT, RR. intervals), neurological-related data (e.g., EEG-related data), genetic-related data, genomic-related data, skeletal data, muscle data (e.g., EMG.related data including surface EMG, amplitude), respiratory data (e.g., respiratory rate, respiratory pattern, inspiration/expiration ratio, tidal volume, spirometry data), thoracic electrical bioimpedance data, or a combination thereof. Some biosensors may detect biological data such as biomechanical data, which may include, for example, angular velocity, joint paths, gait description, step count, or position or accelerations in various directions from which a targeted subject’s movements may be characterized. Some biosensors may gather biological data such as location and positional data (e.g., GPS, RFID-based data; posture data), facial recognition data, kinesthetic data (e.g., physical pressure captured from a sensor located at the bottom of a shoe), or audio/auditory data related to the one or more targeted individuals. Some biological sensors are image or video-based and collect, provide and/or analyze video or other visual data (e.g., still or moving images, including video, MRIs, computed tomography scans, ultrasounds, X-rays) upon which biological data can be detected, measured, monitored, observed, extrapolated, calculated, or computed (e.g., biomechanical movements, location, a fracture based on an X-Ray, or stress or a disease based on video or image-based visual analysis of a subject). Some biosensors may derive information from biological fluids such as blood (e.g., venous, capillary), saliva, urine, sweat, and the like including triglyceride levels, red blood cell count, white blood cell count, adrenocorticotropic hormone levels, hematocrit levels, platelet count, ABO/Rh blood typing, blood urea nitrogen levels, calcium levels, carbon dioxide levels, chloride levels, creatinine levels, glucose levels, hemoglobin Alc levels, lactate levels, sodium levels, potassium levels, bilirubin levels, alkaline phosphatase (ALP) levels, alanine transaminase (ALT) levels, and aspartate aminotransferase (AST) levels, albumin levels, total protein levels, prostate-specific antigen (PSA) levels, microalbuminuria levels, immunoglobutin A levels, folate levels, cortisol levels, amylase levels, lipase levels, gastrin levels, bicarbonate levels, iron levels, magnesium levels, uric acid levels, folic acid levels, vitamin B-12 levels, and the like. In addition to biological data related to the one or more targeted individuals, some biosensors may measure environmental conditions such as ambient temperature and humidity, elevation, and barometric pressure. In a refinement, one or more sensors provide biological data that include one or more calculations, computations, predictions, estimations, evaluations, inferences, deductions, determinations, incorporations, observations, or forecasts that are derived, at least in part, from biosensor data. In another refinement, the one or more biosensors are capable of providing two or more types of data, at least one of which is biological data (e.g., heart rate data and VO2 data, muscle activity data and accelerometer data, VO2 data and elevation data).

In a variation, the at least one sensor 18ⁱ gathers or derives at least one of facial recognition data, eye tracking data, blood flow data, blood volume data, blood pressure data, biological fluid data, body composition data, biochemical composition data, biochemical structure data, pulse data, oxygenation data, core body temperature data, skin temperature data, galvanic skin response data, perspiration data, location data, positional data, audio data, biomechanical data, hydration data, heart-based data, neurological data, genetic data, genomic data, skeletal data, muscle data, respiratory data, kinesthetic data, thoracic electrical bioimpedance data, ambient temperature data, humidity data, barometric pressure data, elevation data, or a combination thereof.

The at least one sensor 18ⁱ and/or its one or more appendices can be affixed to, in contact with, or send one or more electronic communications in relation to or derived from, the subject including a subject’s body, eyeball, vital organ, muscle, hair, veins, biological fluid, blood vessels, tissue, or skeletal system, embedded in a subject, lodged or implanted in a subject, ingested by a subject, integrated to comprise at least a portion of a subject, or integrated into or as part of, affixed to or embedded within, a textile, fabric, cloth, material, fixture, object, or apparatus that contacts or is in communication with a targeted individual either directly or via one or more intermediaries. For example, a saliva sensor affixed to a tooth, a set of teeth, or an apparatus that is in contact with one or more teeth, a sensor that extracts DNA information derived from a subject’s biological fluid or hair, a sensor that is wearable (e.g., on a human body), a sensor affixed to or implanted in the subject’s brain that may detect brain signals from neurons, a sensor that is ingested by an individual to track one or more biological functions, a sensor attached to, or integrated with, a machine (e.g., robot) that shares at least one characteristic with an animal (e.g., a robotic arm with an ability to perform one or more tasks similar to that of a human; a robot with an ability to process information similar to that of a human), and the like. Advantageously, the machine itself may be comprised of one or more sensors and may be classified as both a sensor and a subject. Other examples include a sensor attached to the skin via an adhesive, a sensor integrated into a watch or headset, a sensor integrated or embedded into a shirt or jersey, a sensor integrated into a steering wheel, a sensor integrated or embedded into a video game controller, a sensor integrated into a basketball that is in contact with the subject’s hands, a sensor integrated into a hockey stick or a hockey puck that is in intermittent contact with an intermediary being held by the subject (e.g., hockey stick), a sensor integrated or embedded into the one or more handles or grips of a fitness machine (e.g., treadmill, bicycle, bench press), a sensor that is integrated within a robot (e.g., robotic arm) that is being controlled by the targeted individual, a sensor integrated or embedded into a shoe that may contact the targeted individual through the intermediary sock and/or adhesive tape wrapped around the targeted individual’s ankle, and the like. In another refinement, one or more sensors may be interwoven into, embedded into, integrated with, or affixed to, a flooring or the ground (e.g., artificial turf grass, basketball floor, soccer field, a manufacturing or assembly-line floor), a seat/chair, helmet, a bed, or an object that is in contact with the subject either directly or via one or more intermediaries (e.g., a subject that is in contact with a sensor in a seat via a clothing interstitial). In another refinement, the sensor and/or its one or more appendices may be in contact with a particle or object derived from the subject’s body (e.g., tissue from an organ, hair from the subject) from which the one or more sensors derive or provide information that can be calculated or converted into biological data. In yet another refinement, one or more sensors may be optically-based (e.g., camera-based) and provide an output from which biological data can be detected, measured, monitored, observed, extracted, extrapolated, inferred, deducted, estimated, calculated, or computed. In yet another refinement, one or more sensors may be light-based and use infrared technology (e.g., temperature sensor or heat sensor) to calculate the temperature of an individual or the relative heat of different parts of the individual.

In the variation depicted in FIG. 1, at least one sensor 18ⁱ gathers animal data 14ⁱ from each targeted individual 16ⁱ. Intermediary server 22 receives and collects the animal data 14ⁱ such that collected data has attached thereto individualized metadata, which may include one or more characteristics of the animal data, origination of the animal data, and/or sensor data (e.g., type, operating parameters, etc.). Metadata can also include any set of data that describes and provides information about other data, including data that provides context for other data (e.g., the activity a targeted individual is engaged in while the animal data is collected). Other information, including one or more attributes of the individual from which the animal data originated or other attributes related to the sensor or data, can be added to the metadata or associated with the animal data upon collection of the animal data (e.g., name, height, age, weight, data quality assessments, etc.). In a refinement, source 12 includes computing device 20ⁱ which mediates the sending of animal data 14ⁱ to intermediate server 22, i.e., it collects the data and transmits it to intermediary server 22. For example, computing device 20ⁱ can be a smartphone, smartwatch, or a computer. However, computing device 20ⁱ can be any computing device. Typically, computing device 20ⁱ is local to the targeted individual, although not required. Still referring to FIG. 1, intermediary server 22 provides requested animal data 24 to a data acquirer 26 for consideration (e.g., payment, a reward, a trade for something of value which may or may not be monetary in nature). As used herein, the terms “data purchaser,” “data acquirer,” and “purchaser” are synonymous. In some variations, intermediary server 22 provides raw or processed data, data that has been analyzed, data that has been combined, data that has been visualized, simulated data, and/or reports or summaries about data. Moreover, intermediary server 22 can provide data analysis and other services related to the data (e.g., visualisation, reports, summaries) that may be offered by one or more parties for acquisition (e.g., purchase).

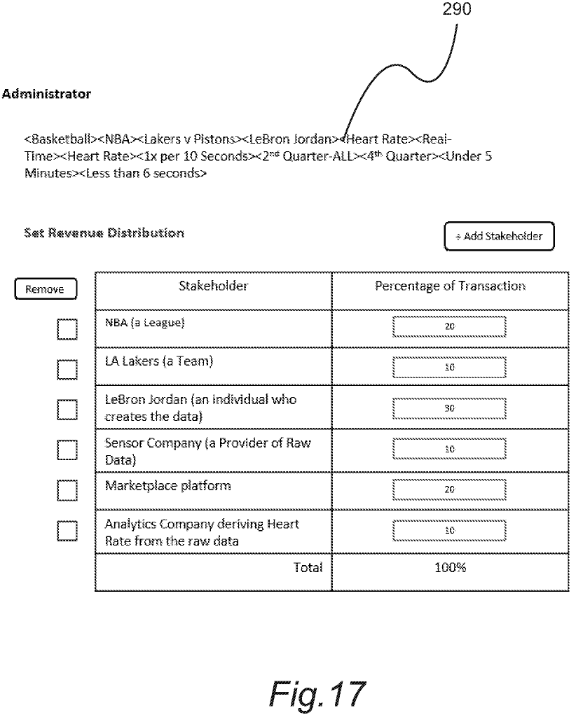

In a refinement, intermediary server 22 synchronizes and tags the animal data with one or more properties (e.g., characteristics) related to the source of animal data. Examples of such properties related to the source of animal data include, but are not limited to, time stamps, sensor type, and sensor settings (e.g., made of operation, sampling rate, gain). Intermediary server 22 can also synchronize the animal data with one or more sensor characteristics, personal attributes, and data types being collected. The intermediary server 22 distributes at least a portion of the consideration to at least one stakeholder 30. The one or more stakeholders can be a user that produced the data, the owner of the data, the data collection company, authorised distributor, a sensor company, an analytics company, an application company, a data visualization company, an intermediary server company that operates the intermediary server, or any other entity (e.g., typically one that provides value to any of the aforementioned stakeholders or the data acquirer). In a refinement, the consideration is distributed in accordance with a revenue share protocol with one or more adjustable parameters that determine the consideration or portion thereof that each stakeholder receives (as shown in FIG. 17).

It should be appreciated that the intermediary server 22 can include a single computer server or a plurality of interacting computer servers. In this regard, intermediary server 22 can communicate with other systems to monitor, receive, and record all requests for animal data to be purchased based on the one or more use cases or requirements. Moreover, intermediary server 22 can be operable to communicate with one or more other systems to monitor, receive, and record all requests for animal data, and provide one or more data acquirers with an ability to search for and make requests for animal data and/or its one or more derivatives by utilizing one or more parameters that are established by the metadata, one or more search parameters, or one or more other characteristics associated with the sensor, data type, targeted individual, group of targeted individuals, or targeted output.

In a variation, intermediary server 22 communicates directly with the source of animal data, as shown by communication links 34 with sensor 18ⁱ or by communication link 36 with computing device 20ⁱ. In a refinement, intermediary server 22 communicates with the source 12 of animal data through a cloud 40 or a local server. Cloud 40 can be the internet, a public cloud, a private cloud utilized by the organization operating intermediate server 22, a localized or networked server/storage, localized storage device (e.g., n terabyte external hard drive or media storage card), or distributed network of computing devices. Typically, source 12 of animal data transmits the animal data wirelessly. However, animal data may be transmitted utilizing a wired connection. In a refinement, source 12 of animal data transmits the animal data to the intermediary server 22 via a hardware transmission subsystem. The hardware system can include one or more receivers, transmitters, transceivers, and/or supporting components (e.g., dongle) that utilize a single antenna or multiple antennas (e.g., which may be configured as part of a mesh network).

As set forth above, the individualized metadata includes origination of the animal data and a targeted individual’s one or more attributes. Examples of such targeted individual’s one or more attributes can include, but are not limited to, age weight, height, birthdate, race, reference identification (e.g., social security number, national ID number, digital identification) country of origin, area of origin, ethnicity, current residence, and gender of the individual from which the animal data originated. In a refinement, the targeted individual’s attributes can include information gathered from medication history, medical records, genetic-derived data, genomic-derived data, (e.g., including information related to one or more medical conditions, traits, health risks, inherited conditions, drug responses, DNA sequences, protein sequences and structures), biological fluid-derived data (e.g., blood type), drug/prescription records, family history, health history, manually inputted personal data, historical personal data, and the like. In the case of human subjects, the targeted individual’s one or more attributes can include one or more activities the targeted individual is engaged in while the animal data is collected, one or more associated groups, one or more social habits, (e.g., tobacco use, alcohol consumption, and the like), education records, criminal records, social data (e.g., social media records, internet search data), employment history, and/or manually inputted personal data (e.g., one or more locations where a targeted individual has lived, emotional feelings). It should be appreciated that various components of the animal data can be anonymized or de-identified. De-identification involves the removal of personal identifying information in order to protect personal privacy. In the context of the present invention, anonymized and de-identified are considered synonymous.

In one variation, the animal data is from a single targeted individual. Such individualized animal data can include a single data set originating from one or more sensors (e.g., a sensor that collects only heart rate or neurological activity to create a single data set; two separate sensors collecting heart rate and neurological activity to create a single data set comprised of both heart rate and neurological activity), or multiple data sets originating from either a single sensor (e.g., a sensor that collects only heart rate, whereby multiple heart rate data sets are created; a sensor that collects both heart rate and sEMG data, whereby one or more heart rate data sets and one or more sEMG data sets are created) or from multiple sensors (e.g., one sensor that collects heart rate and another sensor that collects glucose data, whereby multiple data sets are created from the collected data). In a refinement, a single data set may include multiple data types and/or multiple subjects, and the creation of multiple data sets may be based on only a single individual and a single data type. In another variation, a targeted individual’s data is combined with one or more data sets from one or more other individuals, with either the one or more data sets or individuals sharing at least one or more similar characteristics and provided as a collection of animal data to the data acquirer. In this regard, the intermediary server can populate a data set that is representative of a specific criterion that the data acquirer is looking for. As an example, within an age range of 25-35 year old males, the system can provide data with a 60-40 ratio of 25-30 year old males and 30-35 year old males if desired. In a refinement, the data acquirer defines the criteria that make individuals or the data sets similar. For example, the data acquirer may request DNA or biological fluid data samples from individuals that display a specific genetic trait, but may be dissimilar in other ways (e.g., different age, weight, height). In some variations, composite data is created from multiple data types collected from one sensor or from a plurality of sensors. Classifications (e.g., groups) can be created (e.g., to simplify the search process for a data acquirer, provide more exposure for any given data provider) and may be based on data collection processes, practices, or associations rather than on individual characteristics. For example, a group may be created based upon individuals that collect ECG or PPG sensor data utilizing a specific sensor with specific settings and following a specific data collection methodology. In another example, a group may be created for people who have previously experienced a heart attack. It should be appreciated that any single characteristic related to animal data (e.g., including any characteristic related to the data, the one or more sensors, and the one or more targeted individuals) can be associated with or assigned to one or more groups/classifications or tags. Moreover, the one or more classifications or tags associated with the animal data contribute to creating or adjusting an associated value for the animal data. Examples of classifications or tags include metric classifications (e.g., properties of the subject captured by the one or more sensors that can be assigned a numerical value such as heart rate, hydration, etc.), an individual’s personal classifications (e.g., age, weight, height, medical history), an individual’s insight classifications (e.g., “stress,” “energy level,” likelihood of one or more outcomes occurring), sensor classifications (e.g., sensor type, sensor brand, sampling rate, other sensor settings), data property classifications (e.g., raw data or processed data), data quality classifications (e.g., good data vs. bad data based upon defined criteria), data timeliness classifications (e.g., providing data within milliseconds vs hours), data context classifications (e.g., NBA finals game vs. NBA pre-season game), data range classifications (providing a range for the data, e.g., bilirubin levels between 0.2 - 1.2 mg/dL); and the like. In another variation, some classifications of data may have a greater value than others. For example, heart rate data from people ages 25-34 from Sensor X may have less value than glucose data from people ages 25-34 from Sensor Y. A difference in value may be attributed to a variety of reasons including the scarcity of the data type (e.g., on average, glucose data may be harder to collect than heart rate data and thus less readily available or collectable), the quality of data coming from any given sensor (e.g., one sensor may be providing better quality data than another sensor), the individual or individuals from which the data comes from compared to any other given individual (e.g., an individual’s data may be worth more than another individual’s data), the type of data (e.g., raw AFE data, from which ECG data can be derived, from a group of individuals with certain ethnic characteristics from Sensor X may have more value than only the derived ECG data from the same group of individuals with the same ethnic characteristics from the same Sensor X given that AFE data enables additional non-ECG insights to be derived including surface electromyography data), the derived use cases related to the data (e.g., glucose data can also be used to derive hydration, which may be a more difficult data type to collect than heart-rate based data and therefore more valuable), and the amount or volume of data (e.g., daily heart rate data from 100 people between the ages of 45-54 over the period of 1 year may have more value than daily heart rate data from the same 100 people between the ages of 45-54 over the period of 1 month).

In another variation, collected animal data is assigned to classification (e.g., group) with a corresponding value that may be determined by the system. It should be appreciated that one or more classifications may have a predetermined value, an evolving or dynamic value, or both. For example, a group of data may increase in value as more data is added to the group, as more data within the group is made available, or as demand increases for data from that specific group or may decrease in value as time passes from when the data was created, the data has become less relevant, or demand decreases for data from that specific group. In another refinement, one or more classifications may change dynamically with one or more new categories being created or modified based on one or more purchaser requirements or the input of new information or sources into the system. For example, a new type of sensor may be developed, a sensor may be updated with new firmware that provides the sensor with new settings and capabilities, or one or more new data types (e.g., biological fluid-derived data types) may be introduced into the system from which a data acquirer can search and/or acquire data, or from which a data provider can create new opportunities for value creation. In another refinement, one or more artificial intelligence techniques (e.g., machine learning, deep learning techniques) may be utilized to dynamically assign one or more classifications, groups, and/or values to one or more data sets.

In yet another variation, one or more data quality assessments of the animal data may be provided to a data acquirer or other interested panics as part of the metadata or separately. A data quality assessment provides the animal data’s fitness to serve its purpose in a given context. Factors that are considered when determining data quality include (1) accuracy (or validity or correctness), which occurs when the recorded value is in conformity with the actual value or known range of values; (2) timeliness, which occurs when the recorded value is within the time requirements of duration and latency and not out of date; (3) data consistency (or reliability or lack of conflict with other data values), which occurs when the representation of the data values is the same in all cases; and (4) data completeness, which occurs when all values for a certain variable are recorded (and determines if data is missing or unusable). Additional factors affecting data quality assessments include, but are not limited to, conformity or adherence to a standard format, user feedback rating, and reproducibility of the data. The data quality can be rated or certified in multiple ways, including by one or more experts, by one or more programs written to take into account the one or more factors above to rate the data based on predetermined quality control parameters, and the like. Such a rating can include a predetermined or dynamic data quality scale. In a refinement, the rating and/or certification may be created or adjusted by utilizing one or more artificial intelligence techniques, which takes into account one or more factors.

Advantageously, a value is typically associated with animal data. The value is used for acquiring, buying, selling, trading, licensing, leasing, advertising, rating, standardizing, certifying, researching, distributing, or brokering an acquisition, purchase, sale, trade, license, lease, or distribution of personal identified or de-identified animal data. The value can be monetary or non-monetary in nature. A value that is created for any animal data is inherently assigned to that animal data. Oftentimes, the value is assigned and/or adjusted by the data provider, data owner, or one or more other administrators of data. However, the value may be assigned and/or adjusted by the intermediary server or a third party. In a refinement, the associated value is dynamically assigned and/or adjusted. For example, a specific data set that is assigned a value at a specific time may be assigned with a different value at another point in time, meaning the value of data could change based on one or more factors (e.g., timeliness of data; as an example, in the case of a professional golfer, their heart rate data may have more value to a sports bettor on the 18^{th} green in the final round when he/she is hitting a putt to win the tournament than on the 4^{th} green in the first round when hitting a putt). The intermediary server can be programmed to dynamically assign and/or adjust any given value for any data based upon a variety of factors, classifications, and tags created by the system. In a variation, the same set of animal data may have one or more different associated values. For example, the acquirer of the data, how the data will be used, the duration of the use, the one or more markets in which the data will be used (e.g., the data being used in a single market vs. globally), the timeframe in which the data will be used (e.g., the data being used in real-time vs. at a later date), and the like can all be relevant considerations when assigning different values to the same data, as well as considerations for dynamic assignment and adjustment of a value. In another variation, one or more values are created or adjusted by inputting, at least in part, reference valuation data (e.g., pricing data) from one or more sources (e.g., historical values of sales derived from the monetizationsystem, third party sources that have valued similar data or similar attributes) into one or more models that establish one or more values for one or more data types that are sold by the monetization system. For example, pricing data for heart rate from Player X in League Y of Pro Sport Z may be established by the monetization system by referencing at least a portion of Player X in League Y of Pro Sport Z’s statistical data pricing from one or more third parties, or the historical value of Player X (or individuals similar to Player X) and their similar data within the monetization system as an input to a pricing model that establishes one or more values for the data. In a refinement, the reference valuation data provided may be from one or more dissimilar sets of data. For example, if the monetization system is dynamically establishing pricing for hydration data in Player X in League Y of Pro Sport Z but no pricing for hydration data in a sector (e.g., pro sports) exists, the monetization system may look to other sectors or use cases to establish pricing (e.g., how insurance or fitness-related use cases are pricing hydration compared to captured metrics like heart rate; how other metrics like muscle activity, heart rate, or location data are priced in pro sports and derive a value based upon a set of information). As sales of data sets that have been valued based on other use cases continue, values may dynamically adjust based on demand, scarcity, or other factors. One or more artificial intelligence techniques or statistical models may be utilized to create such value.

In some variations, the system (e.g., via intermediate server 22) may be operable to monitor the life cycle of any given transaction for an individual’s data, including where the data was sent and how, where, and when data was used. Utilizing a technology like blockchain, a data provider or authorized user can view the compete historical tree of that individual’s data, starting from when the data is collected by the system. The system may be operable to monitor animal data and every transaction associated with the data, including details related to any given transaction. This may include verification that the data was collected in a manner purported by the subject, details related to how the data has been used, where the data has been sent, any restrictions attached to the data (e.g., ensuring that use of the data, including any derivative works created, are free and clear from potential future claims), consideration associated with the data, and the like. It can also include enforcement of different types of rights granted to an acquirer when the data is distributed (e.g., exclusivity by territory or data type) and the like. In a refinement, the system may have the ability to enforce restrictions or usage of the data within the blockchain ecosystem. For example, if a party is granted a 15-minute license to the data, the system can ensure that upon expiry of the license, the licensee will be unable to utilize or transfer that data within the blockchain ecosystem.

In another variation, and in cases where one or more data sets derived from the same animal data are distributed to, and utilized by, multiple parties, it may be important for data acquirers to know the manner in which the data has been previously used, as well as the terms associated with that use. In these cases, and utilizing a technology like blockchain, the monetization system may provide functionality (e.g., services) related to the data’s chain of title to ensure that data acquirers obtain and make use of the animal data with an understanding of how, when, and where the data can be used. This may be important to ensure that use of data is free and clear of any future claims. Chain of title can be the official ownership record of any given property such as a subject’s data. In another variation, the monetization system may act as a centralized registry or system that provides one or more records for each type of data distributed and its associated uses. In yet another variation, the monetization system’s data distribution services may also include insurance-related data services (e.g., title insurance related to data usage and derivatives created from distributed data).

In other variations, when an acquirer requests a data type or data set that is not within the intermediary server 22, the intermediary server 22 may send a request to the one or more current users of the system to create the one or more desired data sets or acquire data from one or more third-parties. Alternatively, if the raw (e.g., unprocessed) data to create the requested data exists within intermediary server 22, the intermediary server may process the raw data (e.g., take one or more actions on the data including manipulation, analysis, and the like) to create the acquirer’s requested data. For example, if the system has AFE data derived from a sensor placed on the chest and the request is for ECG data, the system may convert the AFE data into ECG data to fulfill the request. To create the requested data, the intermediary server 22 may use one or more developed tools (e.g., created by the monetization system or operator of the system), incorporate one or more third-party tools housed internally, or send the data (e.g., raw data) to one or more third-party analytics systems, with the intermediary server receiving back the acquirer’s requested data prior to distribution to the acquirer. Upon sending the data to the acquirer, the intermediary server records characteristics of the data provided as part of a transaction. These characteristics of the data include at least one of the following: source(s) of the animal data, time stamps, specific personal attributes, type(s) of sensor used, sensor properties, sensor parameters, sensor sampling rate, classifications, data format, type of data, algorithms used, quality of the data, and speed at which the data is provided (e.g., latency).

In another variation, monetization system 10 provides an alternative to real data sets (e.g., generated by a user or data provider). For example, in the event an acquirer has one or more requirements that may not make it feasible to acquire (e.g., purchase) user-generated data (e.g., the requested data cannot be acquired in a requested timeframe), or an acquirer is unable to afford the acquiring consideration cost of one or more real animal data sets (e.g., the purchase price is too expensive), or the use case required by the acquirer results in one or more data sets that are not found within the system or not obtainable, or the acquirer can only afford a subset of real animal data sets requested, monetization system 10 may provide an option to purchase artificially-generated data (e.g., artificial sensor data) that is created (e.g., generated), derived from, and/or based on at least a portion of real animal data (e.g., real sensor data) and/or its one or more derivatives, which may be generated by monetization system 10 via one or more simulations that conform to one or more parameters (e.g., requirements) set by the data acquirer. In this regard, the one or more parameters the data acquirer selects determines the scope of relevant real animal data that may be utilized as one or more inputs upon which the artificial data is generated, and/or to ensure that the artificial output generated meets the requirements desired by the acquirer. For example, a pharmaceutical company or research organization may want to acquire 10,000 two-hour ECG data sets from at least 10,000 unique males age 25-24 while sleeping, weighing 175-185 pounds that smoke between 10-20 cigarettes per week, having at least one alcoholic drink 2-3 days per week, having a specific blood type with exhibited biological fluid-derived levels, and having a family medical history of diabetes and stroke. The monetization system may only have 500 data sets from 500 unique males that match the minimum requirements of the specific search, so the monetization system can artificially create the other 9,500 data sets for 9,500 unique simulated males to fulfill the pharmaceutical company’s request. The monetization system may use the required parameters and randomly generate the artificial data sets (e.g., artificial ECG data sets) based on the 500 sets of real animal data. The new one or more artificial data sets may be created by application of one or more artificial intelligence techniques that will analyze previously captured data sets that match some or all of the characteristics required by the acquirer. The one or more artificial intelligence techniques (e.g., one or more trained neural networks, machine learning models) can recognize patterns in real data sets, be trained by the collected data to understand animal (e.g., human) biology and related profiles, be further trained by collected data to understand the impact of one or more parameters (e.g., variables, other characteristics) on animal biology and related profiles, and create artificial data that factors in the one or more parameters chosen by the acquirer in order to match or meet the minimum requirements of the purchaser. In a refinement, simulated animal data is generated, at least in part, from collected real animal data In another refinement, one or more statistical models are used. Additional details related to systems for generating simulated animal data and models, as well as examples of how one or more trained neural networks can be utilized within a monetization system, are disclosed in U.S. Pat. No. 62/897,064 filed Sep. 6, 2019; the entire disclosure of which is hereby incorporated by reference and applicable to any artificial data reference in this document. The one or more artificial data sets can be created based on various criteria, including a single individual, a group of one or more individuals with one or more similar characteristics, a random selection of one or more individuals within a defined group of one or more characteristics, a random selection of one or more characteristics within a defined group of one or more individuals, a defined selection of one or more individuals within a defined group of one or more characteristics, or a defined selection of one or more characteristics within a defined group of one or more individuals. Typically, the one or more artificial data sets created via one or more simulations and derived from at least a portion of real animal data share at least one characteristic with real animal data. Based on the purchaser requirements, the monetization system can isolate a single variable or multiple variables for repeatability in creating data sets in order to keep the data both relevant and random. Additionally, the real data and/or its one or more derivatives upon which the simulations are based may be purchased separately, packaged as part of the simulated data acquisition, or utilized as the baseline, at least in part, to create artificial data. In the event an organization requests simulated data, the one or more individuals whose data was in the one or more simulations (e.g., to train the one or more neural networks), at least in part, may receive consideration.

In addition to generating new data sets, the creation of simulated data may also be utilized to extend a previously collected real data set. For example, a system that has access to a specific quantity of data sets for any given activity (e.g., 10, 100, 1000, or more hours of in-match data for Athlete A), which includes different types of data and metadata (e.g., in the context of a sport like tennis, on-court temperature, humidity, average heart rate, oxygenation data, biological fluid-derived data, miles run, swing speed, energy level, shot power, length of points, court positioning, opponent, opponent’s performance in specific environmental conditions, winning percentage, opponent, winning % against opponent in similar environmental conditions, current match statistics, historical match statistics based on performance trends in the match, date, timestamps, points won/lost, score) can extend the data set using one or more artificial intelligence techniques by recreating at least a portion of an event (e.g., a match) in which the given athlete may not have even played and/or generate artificial data for Athlete A within the recreated event (e.g., Athlete A played a 2-hour tennis match with heart rate data captured but a user wants heart rate data for the 3^{rd} hour of a match that was never played and will be played in the future. Therefore, the monetization system can run one or more simulations to create the data). More specifically, one or more neural networks may be trained with one or more of these data sets to understand the biological functions of Athlete A and how one or more variables can affect any given biological function. The neural network can be further trained to understand what outcome (or outcomes) occurred based on the one or more biological functions and the impact of the one or more variables, enabling correlative and causative analysis. Once the neural network within the monetization system has been trained to understand information such as the one or more biological functions of Athlete A within any given scenario including the present scenario, the one or more outcomes that have previously occurred in any given scenario including the present scenario based on the one or more biological functions exhibited by of Athlete A and/or the one or more variables present, the one or more biological functions of athletes similar and dissimilar to Athlete A in any given scenario including scenarios similar to the present scenario, the one or more other variables that may impact the one or more biological functions of Athlete A in any given scenario including scenarios similar to the present scenario, the one or more variables that may impact the one or more biological functions of other athletes similar and dissimilar to Athlete A in any given scenario including scenarios similar to the present scenario, and the one or more outcomes that have previously occurred in any given scenario including scenarios similar to the present scenario based on the one or more biological functions exhibited by athletes similar and dissimilar to Athlete A and/or the one or more variables, an acquirer of data may request one or more simulations to be run, for example, to extend the current data set with artificially generated data (e.g., Athlete A just played 2 hours with various biological data including heart rate captured. An acquirer wants heart rate data for the 3^{rd} hour under the same match conditions, so the system may run one or more simulations to create the data based on previously collected data) or predict an outcome occurring for any given activity (e.g., the likelihood of Athlete A winning the match in the last set vs Athlete B, based on looking only at Athlete A’s data). In a refinement, the one or more neural networks may be trained with multiple animals (e.g., athletes), which may be on a team, in a group, or in competition with one another, and one or more neural networks may be trained with one or more data sets from each animal to more accurately predict one or more outcomes (e.g., whether Athlete A will win the match vs. Athlete B). In this example, the one or more simulations may be run to first generate artificial sensor data based on real sensor data, and then utilize at least a portion of the generated artificial sensor data in one or more further simulations to determine the likelihood of any given outcome.

In another example, an airline may want to determine whether it should extend the mandatory retirement age of its pilots, or a hospital may want to determine whether it should continue to allow a given surgeon to operate past a certain age. By running one or more simulations, the airline or hospital can generate one or more artificial data sets that extend the current one or more data sets collected by the system to facilitate an analysis that enables the airline or hospital to take one or more actions that can determine a probability and/or mitigate a risk. In the airline example, the question may be whether to allow any given n year old pilot (e.g., 65 years old) whose data has been collected by the system an ability to continue to fly past a certain age or while exhibiting specific characteristics which may include either physiological or biomechanical characteristics. More specifically, it may be in the airline’s best interest to determine the biological “fitness” of the pilot and predict future biological fitness rather than mandating a work stoppage (e.g., mandatory retirement) due to an indicator such as a person’s age, as the pilot’s experience could lead to an overall safer flying experience and/or enable more routes to be flown to increase business. Therefore, the system may run one or more simulations for any given pilot utilizing their collected data (e.g., heart/ECG data, age, weight, habits, medical history, biological fluid levels) with various parameters selected (e.g., while sleeping, while flying) and generate one or more artificial data sets (e.g., extending the collected data sets for the pilot and creating artificial sensor data to see the pilot’s heart activity from future ages 66-80 to determine biological “fitness” and “fitness for flying” as the pilot ages). In the case of the hospital, the question may be whether to allow any given surgeon to continue to operate past a certain age or while exhibiting specific characteristics which may include either physiological or biomechanical characteristics, with the benefit being able to utilize the surgeon’s experience which could lead to saving more lives.

In a refinement, simulations can provide one or more probabilities or predictions related to a future outcome occurring. For example, if an airline wants to know the likelihood of whether or not any given pilot exhibiting specific physiological characteristics will have a heart attack while flying a plane, one or more simulations that utilize at least a portion of the pilot’s animal data can be run, the output of which can be used to determine the probability of the occurrence happening or make a prediction related to a future event. In another example, if an insurance company wants to know the likelihood of whether or not any given person with specific characteristics (e.g., age, weight, height, genetic makeup, medical conditions) will experience one or more physical ailments (e.g., stroke, diabetes, virus) within a given period of time (e.g., 24 months), one or more simulations that utilize at least a portion of real animal data can be run with these characteristics as one or more inputs, the output of which can be used to determine the probability of the occurrence happening. In another example, if a pharmaceutical company wants to better understand the probability of an existing drug having a specific effect on one or more individuals with specific characteristics, the monetization system can run multiple simulations (e.g., 10, 100, 10000, or more) to determine the probability of an occurrence happening. In yet another example, if a team wants to know the likelihood of whether Player A on a sports team will make the next shot based on exhibiting specific physiological characteristics and other collected data, one or more simulations that utilize at least a portion of Player A’s animal data can be run, the output of which can be used to determine the probability of the occurrence happening.

In a variation for creating one or more simulated data sets, existing data with one or more randomized variables is re-run through one or more simulations to create new data sets not previously seen by the system. Utilizing this method, one or more probabilities related to one or more outcomes can be examined. For example, when the monetization system has data sets for a specific individual (e.g., athlete) and a specific event (e.g., match the athlete has played), the system may have the ability to re-create and/or change one or more variables within the data set (e.g., the elevation, on-court temperature, humidity) and re-run the one or more events via one or more simulations to generate a simulated data output for a specific scenario (e.g., For example, in the context of tennis, an acquirer may want 1 hour of Player A’s heart rate data when the temperature is at or above 95 degrees for the entirety of a two-hour match. The system may have one or more sets of heart rate data at different temperatures (e.g., 85, 91, 94) as well as previously described inputs for Player A in similar conditions as well as other similar and dissimilar athletes in similar and dissimilar conditions. Heart rate data for Player A at or above 95 degrees has never been collected so the system can run one or more simulations to create it, and then utilize that data in one or more further simulations. In another example, the acquirer may want the likelihood that Player A will win the match. In a refinement, the system may also be programmable to combine dissimilar data sets to create or re-create one or more new data sets. For example, an acquirer may want 1 hour of Player A’s heart rate data when the temperature is above 95 degrees for the entirety of a two-hour match for a specific tournament, where one or more features such as elevation may impact performance. While this data has never been collected in its entirety, different data sets can comprise the requested data (e.g., one or more data sets from Player A featuring heart rate, one or more data sets from Player A playing tennis in temperatures above 95° F., one or more data sets at the required tournament with requested features such as elevation). The system may identify these requested parameters within the data sets and across data sets and run one or more simulations to create one or more new artificial data sets that fulfill the acquirer’s request based on these dissimilar sets of data. In a variation, the dissimilar sets of data that are used to create or re-create one or more new data sets may feature one or more different subjects that share at least one common characteristic with the targeted individual (which can include, for example, age range, weight range, height range, sex, similar or dissimilar biological characteristics, and the like). Using the example above, while heart rate data may be utilized for Player A, the system may utilize another one or more data sets from Players b, c, d, which have been selected based upon its relevancy to the desired data set (e.g., some or all of the players may have demonstrated similar heart rate patterns to Player A; sonic or all of the players have similar biological fluid-derived readings to Player A; some or all of the players may have data sets collected by the system that feature tennis being played in temperatures above 95 degrees). These one or more data sets may act as inputs within the one or more simulations to more accurately predict Player A’s heart rate under the desired conditions.

In another method for simulated data, randomized data sets are created, with the one or more variables selected by the system rather than the acquirer. This may be particularly useful if, for example, an insurance company is looking for a specific data set (e.g., 1,000,000 smokers) amongst a random sample (e.g., no defined age or medical history, which may be selected at random by the system). In a refinement, one or more artificial data sets are created from a predetermined number of individuals picked at random by the system.

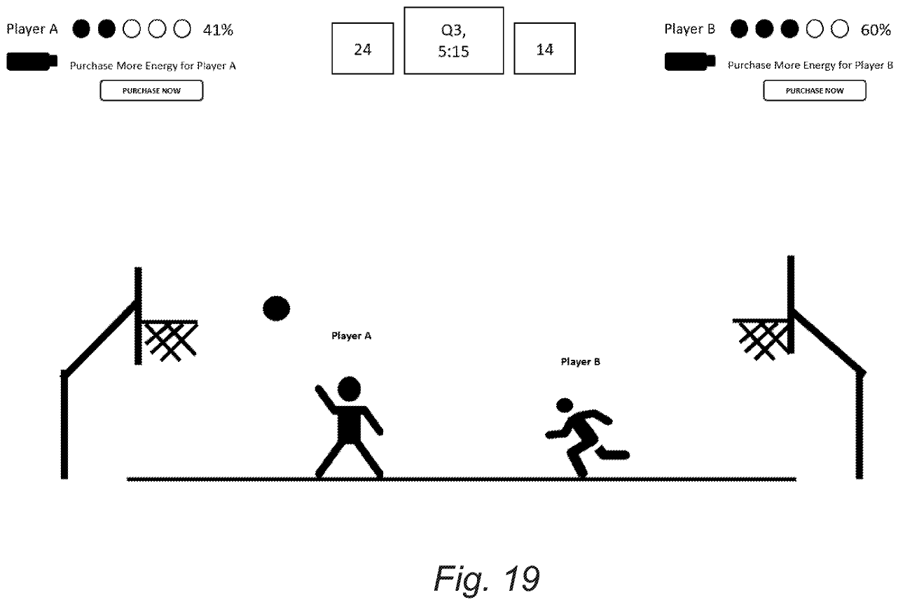

In another example, data derived at least in part from real animal data may be acquired as part of, or utilized within, a video game or game-based system. A video game or game-based system may be played within a variety of consoles and systems provided including traditional PC gaming (e.g., Nintendo, Sony PlayStation), handheld gaming, virtual reality, augmented reality, mixed reality, and extended reality. The video game or game-based data, which may be derived from one or more simulations and/or created artificially based upon at least a portion of the animal data, can be associated with one or more characters (e.g., animals) featured as part of the game. The characters may be based on animals that exist in real life (e.g., a professional soccer athlete in real life may have a character that portrays themselves in a soccer video game) or artificially created, which may be based on, or share, one or more characteristics of one or more real animals (e.g., a soccer player within a game shares a jersey number, a jersey color, or biological feature as a human soccer player). The system may enable a user of a video game or game-based system to purchase data or purchase a game that utilizes at least a portion of real data within the game. In a refinement, the animal data purchased within the game may be artificial data, which may be generated via one or more simulations. This data may be utilized, for example, as an index for an occurrence in the game. For example, a gamer may have the ability to play against a simulated version of a real-world athlete in a game utilizing the athlete’s “real-world data,” which may include the athlete’s real-world biological data or its one or more derivatives. This may mean that, for example, the real-world athlete’s “energy level” data that has been collected over time is integrated into the game. In one specific example, as the length of a match within a video game goes on, or the distance the simulated athlete within the video game has run, their “energy level” within the video game may be adjusted and impacted based upon a real athlete’s collected real-world data. The real-world data can indicate how fatigued an athlete may get based on distance run or length of any given match. This data also may be utilized, for example, to gain an advantage within the game, which may include an ability to run faster, jump higher, have longer energy life, hit the ball farther, etc. FIG. 19 illustrates one example of a video game whereby a user can purchase a type of artificially-generated animal data (e.g., “energy level”) based at least in part on real animal data to provide the user of the video game with an advantage. In another example, the in-game artificial data, which is derived from or shares at least one characteristic with animal data, may also provide one or more special powers to the one or more subjects within the game, which may be derived from one or more simulations. In another refinement, one or more individuals that provide at least a portion of their animal data and/or its one or more derivatives to a video game or game-based system may receive consideration in exchange for providing that data. For example, a star tennis player may provide his or her biological data to a video game company so that a game user can play as, or against, a virtual representation of that star tennis player. In this situation, the user may pay a fee to the video game company for access to the data or a derivative thereof (e.g., artificial data generated based upon at least a portion of the real animal data), a portion of which may go to the star tennis player. Alternatively, the video game company may pay a license fee or provide other consideration (e.g., a percentage of game sales or data-related products sold) to the athlete for the use of the data within their game. In another example, the video game company can enable one or more bets/wagers to be placed on the game itself (e.g., between the user and the star tennis player) or proposition bets within the game (e.g., micro bets based upon various aspects within the game). In a refinement, the one or more prop bets are based upon at least a portion of the animal data and/or its one or more derivatives (including simulated data). In this situation, the user and/or star tennis player may receive a portion of the consideration from each bet placed, the total number of bets, and/or one or more products created, offered, and/or sold based upon at least a portion of the data.

Although the present invention is not limited to any particular application for using simulated data, such data can be used as a baseline or input to test, change and/or modify sensors, algorithms, and/or various hypotheses. This artificial data can be used to run simulation scenarios, which range from training to improving performance. A potential reason for using artificial data based on real data is that the costs could be significantly lower for artificial data than for real data. Real data may have one or more specific rights associated to it whereas artificial data that is based on the patterns and knowledge of real data may have no (or limited) rights attached and therefore can be acquired (e.g., purchased) at a much lower cost. Moreover, data generated from one or more simulations can be used for a wide array of use cases including as a control set for identifying issues/patterns in real data, as an input in further simulations, or as an input to artificial intelligence or machine learning models as test sets, training sets, or sets with identifiable patterns. For example, a data set created based on real data from a particular individual can be modified using this system to introduce deviations in the data corresponding to characteristics like fatigue or rapid heart rate changes. With this modified data, simulations can be run to see how the individual will perform in, as an example, high-stress situations or in certain environmental conditions (e.g., high altitude, high on-court temperature). Such simulations can be particularly useful in fitness applications, insurance applications, and the like. In the case of a human (e.g., athlete) or other animals, the system may establish the patterns between biological metrics (e.g., heart rate, respiration, location data, biomechanical data), and the likelihood of an occurrence happening (e.g., winning a particular match). In this situation, the monetization system can calculate probabilities of certain conditional scenarios (e.g., “what-if” scenarios and likely outcomes).

As set forth above, the intermediary server receives the animal data in raw form or processed form. In this regard, the intermediary server can take one or more actions upon the animal data. For example, the intermediary server can operate on the animal data by implementing at least one action selected from normalizing the animal data, associating a time stamp with the animal data, aggregating the animal data, applying a tag to the animal data, storing the animal data, manipulating the animal data, denoising the animal data, enhancing the animal data, organizing the animal data, analyzing the animal data, synthesizing the animal data, replicating the animal data, summarizing the animal data, anonymizing the animal data, visualizing the animal data, synchronizing the animal data, displaying the animal data, distributing the animal data, performing bookkeeping on the animal data, and combinations thereof.

In another embodiment, the system may be utilized as a tool to test, establish, and/or verify the accuracy, consistency, and reliability of a sensor or connected device. Sensors that produce a similarly labeled output (e.g., heart rate) my use different components (e.g., hardware, algorithms) to derive their output. This means that, for example, an output like heart rate from one device may not be the same as heart rate from another device. The system’s ability to bypass native applications and act upon the data, including normalizing and/or syncing the data, ensures a user has the ability, if desired, to do a relative “apples-to-apples” comparison and compare each sensor output and their corresponding hardware/firmware and algorithm(s) that derive each output (e.g., raw data, processed data), while providing context for the data (e.g., the activity upon which the data was collected) and eliminating other variables (e.g., transmission-related, software-related) that may impact the output. Testing and comparing each sensor or connected device hardware, algorithm(s), or output impartially (e.g., against a designated standard) ensures quantifiable results. An ability to obtain quantified results for each sensor type and its corresponding components enables a user to select a particular sensor and/or algorithm for participants of a given group based upon any given requirement or use case (e.g., activity) while removing key sensor-related variables typically found in studies that are using different or inferior hardware components (e.g., different sensors capturing the “same” output) or different algorithms. This process removes potential variables that may impact a result and ensures a trust in the data by a user. Similarly, it provides acquirers with a quantifiable way to select one or more sensors and/or place a premium value on any given output. It also enables the system to place a premium value on any given output.

Another aspect of the monetization system is the collection of consideration for the animal data. Upon sending animal data to the user, the intermediary server monitors and/or records collection of the consideration for the animal data that was provided. The collection of consideration may occur simultaneously as the transaction occurs or at a later time. In a refinement, collection may occur prior to the sending of any data to the acquirer. Advantageously, the animal data can be offered on a marketplace or other medium for such sale or acquisition of animal data. Typically, the data acquirer (e.g., purchaser) buys or acquires at a price or value the data provider creates. The marketplace can be populated with data from any type of individual with a variety of characteristics (e.g., age, height, weight, hair color, eye color, skin tone, etc.) with any or no pre-existing condition (e.g., diabetes, hypertension, kidney disease), from any location (e.g., on earth, in space), using any type of sensor that collects data doing any imaginable activity. In a refinement, the monetization system may prescribe the type of data that is needed in the marketplace based on likely demand that is determined from things such as search results by data acquirers, and create a call to action for the data providers to supply specific data for which they will receive a fee once the data has sold. In another refinement, the data acquirer can define the criteria of the one or more individuals, the one or more locations, the one or more sensors, the one or more activities, and whether video of the one or more activities is required, and set a price for that data for the data providers to accept or decline. The marketplace will enable a data acquirer to collect the data from the data providers who have accepted the offer either in real-time or within a deadline that is set by the data acquirer. For example, if a sensor manufacturer is wanting to collect data from n number of individuals and the sensor manufacturer wants those individuals to follow specific instructions (e.g., activity or movement), the sensor manufacturer can initiate a video conference to show each individual what to do (e.g., either live or delayed basis). Advantageously, this process may enable the data acquirer to leverage the artificial intelligence and machine learning capabilities of the monetization system to determine whether the data being collected by each individual is in tact viablc data or not, rather than waiting until the entire data set is collected. If for example, the sensor manufacturer neither needs the data in real-time nor needs to explain how to collect data, then the individual data providers can collect the data on their own time within the deadline and upload it via the monetization system. The marketplace will also incorporate a feedback mechanism whereby the data acquirers can rate, for example, each individual’s quality of data collection, how accommodating they are, reliability, timeliness and diligence in returning any sensors or hardware as well as other attributes. Some of the components of the feedback ratings will be driven by the monetization system where applicable such as timeliness of data submission.

In a variation, the data acquirer can set a price or value for the animal data or place one or more bids to acquire the animal data. In another variation, the monetization system determines, at least in part, the value of the animal data based on one or more variables (e.g., time, demand, scarcity, sensor the data is derived from, quantity). In a further refinement, the data acquirer can make one or more requests/bids for data from one or more subjects that have or use one or more characteristics requested by the data acquirer (e.g., specific personal attributes, type of data, type of sensor used). The data acquirer may or may not know the identity of the one or more subjects depending on the request. In another refinement, the data provider can bid for a data acquirer’s request for data.

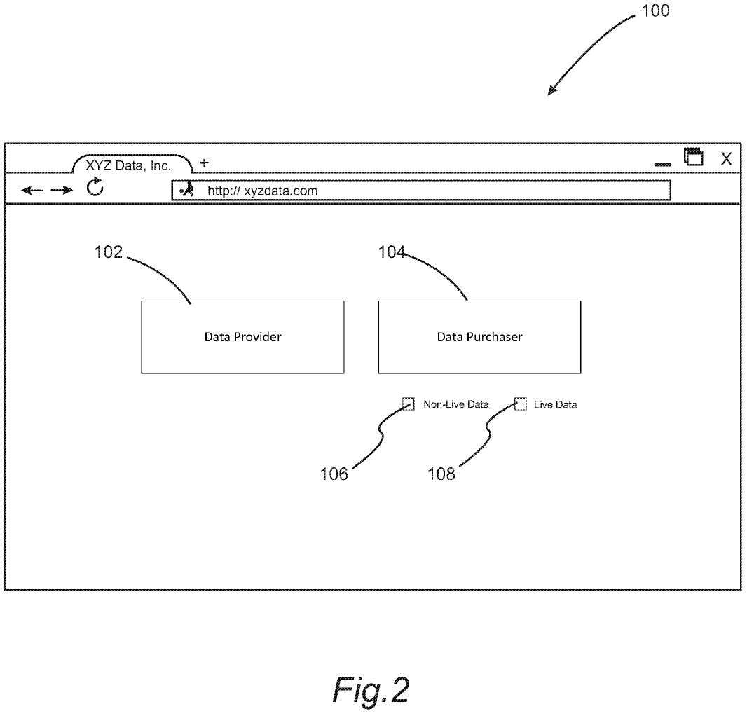

FIGS. 2 to 17 illustrate the functionality of the monetization system of FIG. 1 that can be deployed in a web page or in a window for a dedicated program or computing device (e.g., smart device) application. FIG. 2 provides an illustration of a window 100 through which a user (e.g., data acquirer, data provider) can interact with the monetization system set forth above. The term “window” will be used to refer to a web page and/or window for a program or computing device (e.g., phone, tablet, etc.) application. Window 100 includes a control element 102 that is selected for the user to identify as a data provider or a control element 104 for the user to identify as a data acquirer. Each of control elements 102, 104 are depicted as “buttons.” It should be appreciated that for each of the control elements depicted in FIGS. 2 to 17, control elements such as selection boxes, dropdown lists, buttons, and the like can be used interchangeably. In a refinement, one or more control elements may be replaced by one or more verbal, neurological, physical, or other communication cues, including communicating the command using a voice-activated assistant, communicating the command with a physical gesture (e.g., finger swipe or eye movement), or neurologically communicating the command (e.g., a computing device like a brain-computer interface may acquire one or more of the subject’s brain signals from neurons, analyze the one or more brain signals, and translate the one or more brain signals into commands that are relayed to an output device to carry out a desired action. Acquisition of brain signals may occur via a number of different mechanisms including one or more sensors that may be implanted into the subject’s brain). This can also apply to elements such as login credentials required to access the monetization system. The data provider and the data purchaser can each independently be an individual (e.g., person) or entity (e.g., administrator of a company, organization, or group) representing one or more individuals, or one or more individuals or entities. Window 100 also includes selection box 106 by which a user can select non-live data (e.g., previously collected) or selection box 108 by which a user can select live data. Live data includes data that is collected in real time, near real-time, or in a timeframe in which the data being collected is made available while the activity/event, or continuation of the activity/event, is still occurring. In a refinement, selecting box 108 may also enable a user to search for and acquire at least a portion of non-live data.

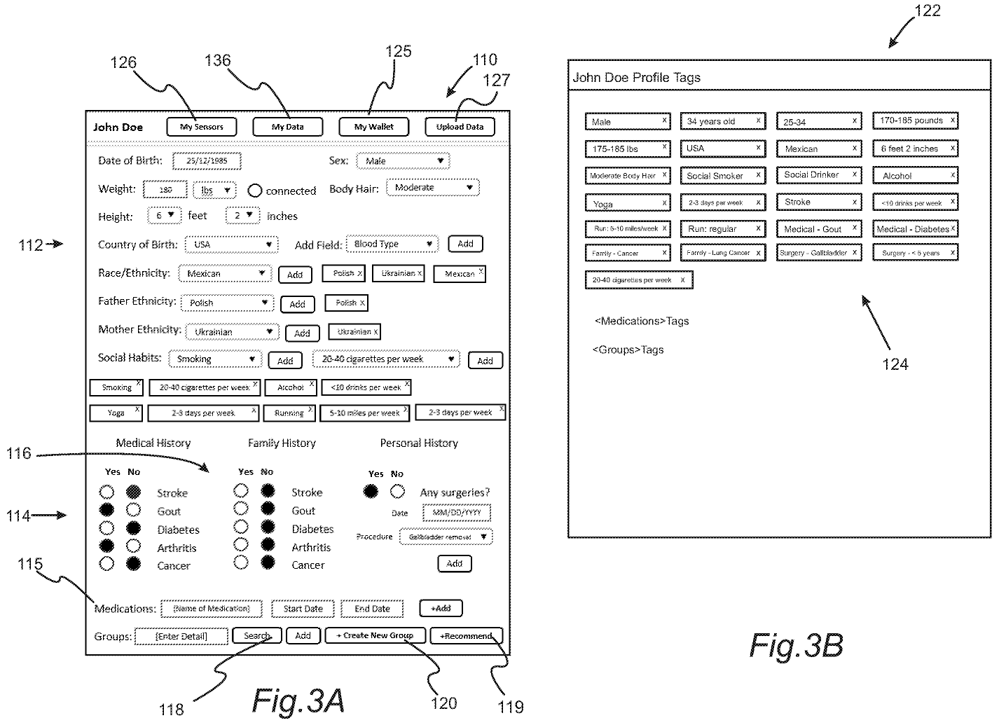

FIG. 3A provides an illustration of a window presented to a data provider after the selection of control element 102 in FIG. 2 is made. Prior to FIG. 3A, login credentials may be provided. Window 110 is an initial setup page for an individual. Window 110 includes section 112 where a creator of data or administrator/manager (e.g., user) can enter in a subject’s various individual attributes. In the case of a human, this includes age, height, personal history, social habits, and the like. One or more fields provided by the system may be added by the user (e.g., data provider) should the user want to provide additional information to create more targeted searches (e.g., blood type) for a data acquirer. One or more photos or visual representations of the user may also be uploaded and made available via button 127. Window 110 also includes section 114 for entering medical history information, section 115 for entering medication history, and section 116 for entering family history. The example fields provide only a sample list of the potential input parameters. Other types of personal information may also be included or uploaded including personal history (e.g., surgeries, broken bones, abuse, other illnesses), more granular data including genetic/genomic information related to an individual (e.g., one or more data sets related to an individual’s DNA sequences, protein sequences and structures, RNA sequences and structures, gene expression profiles, gene-gene interactions, DNA-protein interactions, DNA methylation profiles), and the like. The user may also upload additional personal information such as biological fluid data, which can be gathered utilizing one or more sensors and can include information derived from blood (e.g., venous, capillary), saliva, urine, and the like. The one or more gathered data types can be one or more searchable parameters created by the system. In a refinement, one or more types of biological fluid data may be combined into one or more groups, including groups related to one or more tests or panels (e.g., complete blood count, comprehensive metabolic panel, renal function panel, electrolytes panel, basic metabolic panel, hepatitis panel, and the like) and test categories (e.g., information related to estradiol levels, prolactin levels, progesterone levels, DHEA-sulfate levels, and follicle stimulating hormone levels may be categorized as part of a female reproductive health test) to enable more efficient search and data acquisition parameters. This may be useful, for example, if an acquirer is interested in examining one or more biological components or functions (e.g., liver and kidney health) across one or more subjects that utilize the same data inputs. In another refinement, the monetization system may be operable to enable one or more search functions (e.g., including creation of one or more groups) based upon variations within the data. For example, an acquirer may have the ability to search for individuals that exhibit variations or ranges within specific biological traits (e.g., blood sugar levels of less than 100 mg/dL, potassium levels between 5.1 mEq/L and 6.0 mEq/L, males with a red blood cell count range of 4.9 to 5.8 million cells per microliter of blood, and the like). Similar to other collected animal data, biological fluid information may be information an acquirer is interested in obtaining either as complimentary information related to a data set (e.g., a person acquiring heart-based data may want to use biological fluid-related data from an individual as a parameter, such as an acquirer who wants ECG data from individuals that have a low white blood cell or red blood cell count), or as data itself (e.g., the raw or processed information gathered from the one or more sensors and derived from biological fluid as one or more data sets). In another refinement, the user may upload artificial data that shares at least one characteristic with real biological animal data (e.g., computer vision data).

Note that FIG. 3A displays only a sample of potential personal parameters that the system may provide, at least some of which can be tunable parameters and may be added as one or more searchable parameters by the system. Control element 119 provides one or more recommended groups for the user to join based upon the information provided to the monetization system (e.g., individual information, sensor information, activity information, data information). Finally, control element 118 can be used to search for one or more terms (e.g., group name, one or more individual or sensor characteristics, activity in which the sensor data is collected) to associate the data provided to window 110 to a previously created group while control element 120 is used to create a new group. In a refinement, one or more groups are automatically assigned or associated to an individual’s profile by the system based on inputted data. FIG. 3B shows a listing 122 of tags 124 that are created in association with the selections made and data inputted in window 110. With each characteristic inputted, a tag is created by the system (column located on the right) as depicted in FIG. 3B. These tags may be exact matches based on data inputs (e.g., “male” if the subject is male) or they may be created based on inferences or created classifications so that a data acquirer can more easily search across the data based on desired parameters. For example, if a user is a smoker of 20-40 cigarettes a week, the monetization system may create a tag called “social smoker,” which is inferred based on the number of cigarettes smoked per week (and the monetization system’s determination that 20-40 cigarettes is considered social). Tags may also be retroactively or dynamically created based upon requests from the data acquirer or other considerations (e.g., demand based on an increased number of searches may result in new tags being created for previously collected data). A user can also add themselves to a group or create a group which will create additional tags for an individual. These groups can represent a number of different linking characteristics or indicators. For example, a group can be a team an individual is associated with. A group can be two or more people that utilize specific processes and methodologies to more accurately collect data (which may be deemed to have more value than other data collection processes and methodologies). Association with the latter example group may mean one or more data sets associated with this group have more value to a data acquirer if the data acquirer is looking to acquire data utilizing the group’s specific processes and methodologies. In a refinement, one or more associations (e.g., tags, groups) may be assigned by the system to any individual or data set automatically by utilizing one or more artificial intelligence techniques. In another refinement, the monetization system may be programmed to reject a user’s ability to assign one or more groups to any given user.

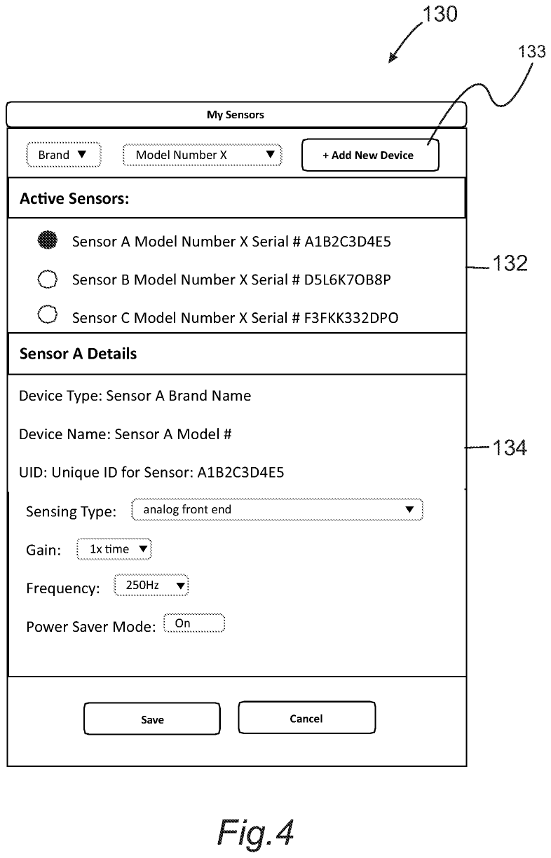

FIG. 4 is an illustration of a window that provides sensor information. Window 110 of FIG. 3A includes control element 126 labeled “My Sensors” at the top. Actuation of control element 126 causes page 130 to be displayed which shows the user’s active sensors 132 (e.g., sensors that are used for data collection) and enables the user to view the sensor settings/parameters 134. In some cases, the user will have the ability to change the one or more sensor settings for the one or more sensors within the platform by enabling the monetization platform to communicate directly with the one or more sensors. Control element 133 enables one or more new sensors that collect data from the user to be added. Adding sensors can occur in a number of ways. For example, by clicking control element 133, the monetization system may be programmed to take one or more actions which could include scanning for, detecting, adding, and/or pairing with one or more new sensors, as well as assigning one or more new sensors to an individual. However, the present invention is not limited by the ways a device can be added.

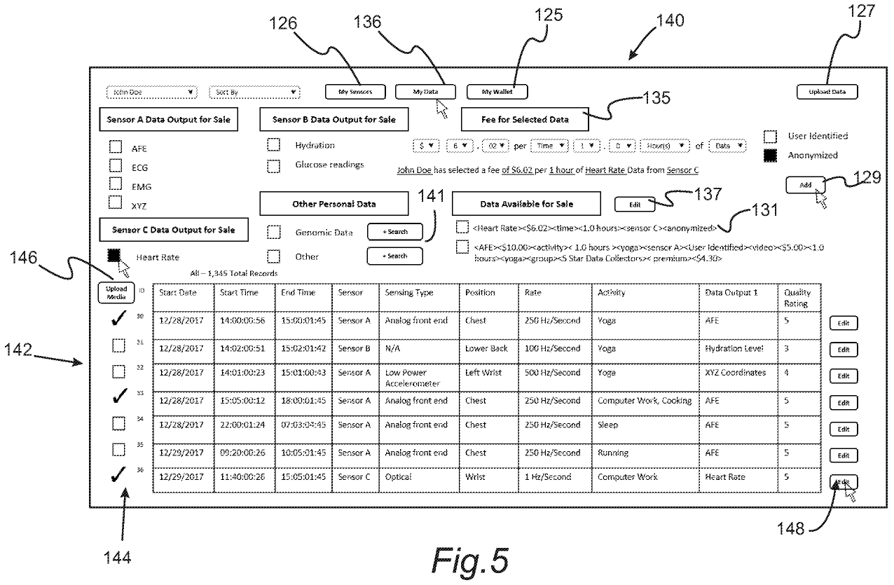

FIG. 5 is an illustration of a window for a user to manage their data, including the one or more sensors that were used to capture the data within FIG. 5, the associated metrics that have been collected by the monetization system via the one or more sensors, metadata associated with the collected data, the one or more data types that can be made available for sale, and the user’s ability to set a price for any data type from any selected sensor or data set. Actuation of the control element 136 labeled “My Data” of FIG. 3A displays window 140 which shows the sensors that are active and the associated metrics being collected by the sensors. If the user is a manager of multiple users, the managing user has the ability to select information for display related to one or more managed users. In a refinement, window 140 may also include data from sensors that are not active, which may also be made available for sale. FIG. 5 also shows additional data 141 that may be made available for sale. Data 141 can include data derived from sensors and captured by the monetization system, or uploaded via element 127 and made available for acquisition by a data provider. Window 140 also shows data records 142 that have been collected with relevant data characteristics including IDs, time stamps, sensor settings, and the like. The user can also create the acquisition cost (e.g., price) that the user will charge for their data by one or more parameters including sensor and data type. In a refinement, the user can create the data acquisition cost based on any parameter including time, activity from which the data was collected (e.g., the cost of engaging a particular activity for a user may increase the cost of the data), and the like. The user can set the parameters in window 140. Consideration value may be established by the user via element 135. In a refinement, element 135 can include one or more fields that enable a user to set a value based upon more granular information (e.g., creating a value by activity). For example, a user may establish a higher value for one activity (e.g., engaging in yoga for 1 hour) compared to another activity (e.g., sleeping) using the same sensor. The user can also choose whether they want their data to be made available with their identification attached or anonymously. After establishing the fee for selected data 135 and selecting control element 129, the acquisition terms established by the user are displayed 131. The acquisition terms established by the user can be adjusted or edited any time by selecting control element 137. In a refinement, a user may also have an ability to attach one or more ancillary items to the data to add more value to the data. For example, if a user has video of the activity upon which the data was collected, the video can be uploaded and associated with any specific data set by clicking selection element 144 (e.g., a selection box) on the left-hand side and clicking on control element 146 labeled “Upload Media.” Similarly, one or more photos of the one or more sensors on the user’s body, or other media associated with the data, may also be uploaded. If the environment in which the data is collected (e.g., humidity, temperature, elevation) or other conditions that may have an impact on the data are known (e.g., skin color/tattoos for certain optical sensors), that information may also be added, with the system operable to identify one or more common characteristics between the collected data sets (e.g., time stamps, location) in order to link data sets together. In a refinement, social data or other forms of data associated with the user or group of users that may provide context or value to the collected sensor data may be uploaded. In another refinement, a premium may be applied to one or more data sets based on one or more tags associated with the data, which may be assigned by the system dynamically. For example, if an individual’s heart rate data is associated with a specific sports league, or an individual is associated with a specific group that collects data utilizing a process that enables for more accurate data to be collected, the system may assign a premium value to the one or more requested data sets. The assignment of a premium value may occur dynamically based on one or more factors (e.g., a new group is created at a later time in which a data set is assigned a premium value; demand for a data set increases over time so that a data set which originally did not have a premium value now has a premium value). In some cases, the premium may be viewable by the user in area 131. In other cases, the premium may not be viewable to the user (e.g., in the event the premium is not allocated to the user, or if the premium is dynamically assigned at a later date). In another refinement, more than one premium may be applied or associated to any given data set. Multiple premiums may be associated to given a data set in area 131 based on one or more tags or considerations created or determined by the system, which may occur at the same time or at different times (e.g., a premium may be assigned at a later time based on dynamic factors including increased demand at a later date, as well as tags created dynamically or automatically at a later date that have a premium value associated).

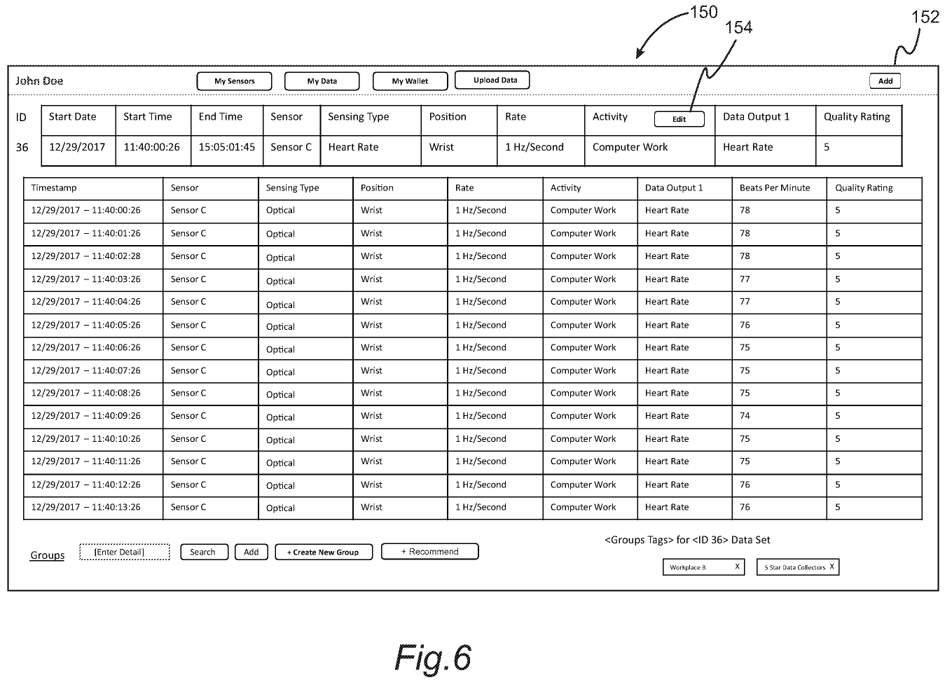

FIG. 6 depicts a window providing additional detail related to any given collected data set, as well as an ability to modify one or more aspects any given data set. If a user wants a more granular view of the data, actuation of control element 148 in FIG. 5 causes window 150 in FIG. 6 to be displayed. If the user is a manager of multiple users, the managing user has the ability to select information for display related to one or more managed users, as well as other characteristics of the one or more managed users or data. FIG. 6 shows the details of the data that an individual data provider (e.g., user) has collected. It should be appreciated that window 150 lists sensor type, position of the sensor, sampling rate, activity of the subject being measured, sensor output, and an assessment of quality. Note that FIG. 6 displays only a sample of potential information that the system may provide, all of which are tunable parameters. In some cases, the system may be programmed to enable additional information (e.g., metadata, notes) related to the sensor or collected data to be added once the data has entered the system via element 152, which may be made available as part of any given data acquisition. In addition, the system may be programmed to identify one or more details related to the metadata that may be edited by the user or administrator (e.g., data manager). For example, the administrator may have the ability via actuation element 154 to edit or add certain types of descriptive information (e.g., activity). This ability may be removed or added depending on the user or the data set, or blocked or enabled by the monetization system based on the metadata provided. Moreover, the user has the ability to assign additional Group tags to a specific data set or receive recommended group tags from the monetization system in the event a user wants to be able to further categorize and tag the data. In a refinement, the monetization system may be programmed to reject a user’s ability to assign one or more groups to any given data set (e.g., if a user does not fit the profile or the collected data does not meet the requirements of the one or more groups as determined by the monetization system or administrator). The monetization system may also assign tags automatically to the data without requiring any input from the data provider. For example, by looking at the metadata, the monetization system may be operable to identify groups of data that were collected together at the same time and under the same conditions.

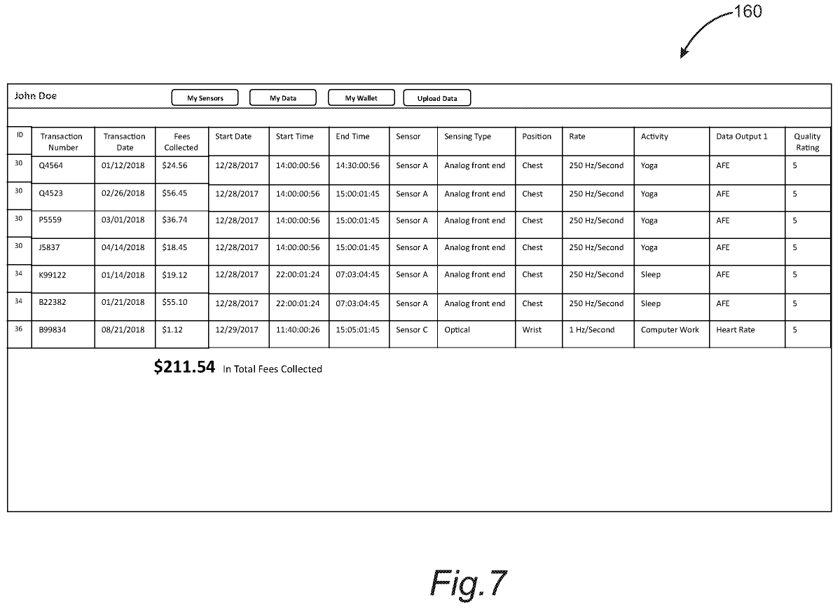

FIG. 7 is a summary page of the consideration collected by the system on behalf of the user (in this example, John Doe). Actuation of control element 125 labeled “My Wallet” of FIG. 3A displays window 160 which provides a summary page that displays the fees collected for any individual data provider. The total purchase price, which may include one or more premium values placed by the system based on the one or more tags associated with the data for each set of data, may be different than the fee collected, as the consideration or total purchase price received may be distributed to one or more additional parties (e.g., sensor manufacturer, analytics company). As described in summary page 160, multiple stakeholders may have claim to some form of revenue for any single transaction, including the individual provider/creator of the data or a group administrator. This page simply displays the fee each data provider receives. In addition, it should be appreciated that an individual can sell the same data set to multiple users at different purchase prices and at different times. The monetization system will also provide a purchaser with the ability to purchase the data exclusively, or set custom parameters or restrictions (e.g., territorial rights, usage rights) around the purchaser’s specific use.

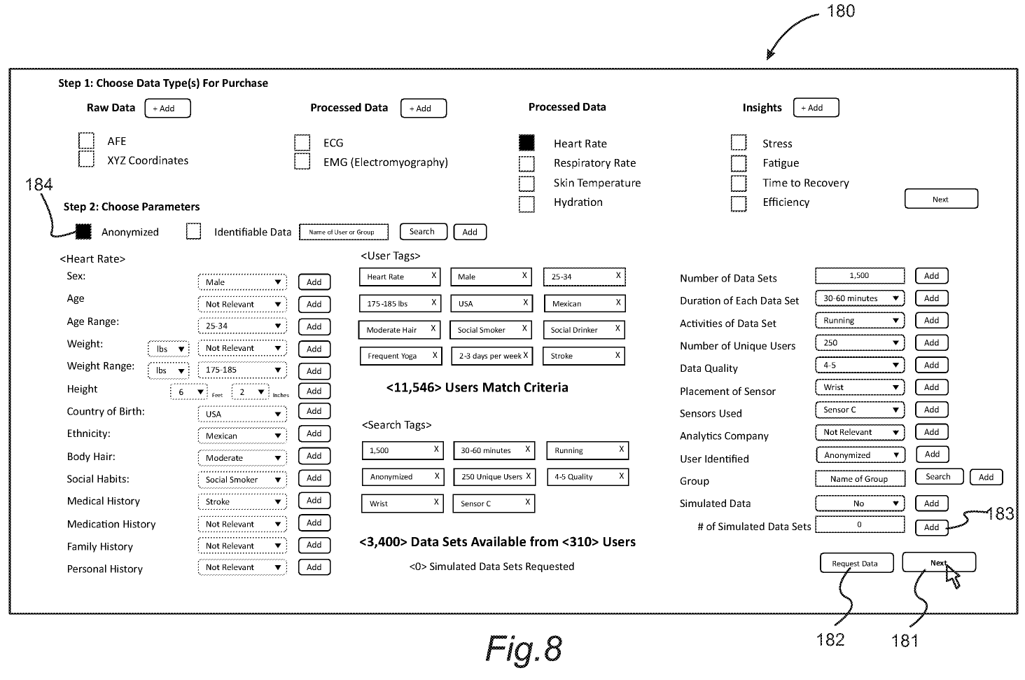

FIG. 8 illustrates the scenario when a data acquirer requests non-live data (e.g., historical data sets). A data acquirer of both live and non-live data can be represented by a wide range of profiles including financial trading companies, sports teams, sports broadcasters, sports betting-related organizations, municipality groups (e.g., police, firefighters), hospitals, healthcare companies, insurance organizations, manufacturing companies, aviation companies, transportation companies, pharmaceutical companies, military organizations, government entities, automobile companies, telecom companies, food & beverage organizations, ICT organizations, elderly care organizations, construction companies, research institutions, oil & gas companies, personal health companies, analytics organizations, other technology companies, individuals, and the like. When a data acquirer selects control element 104 indicating the user is a data acquirer and selection box 106 in window 100 of FIG. 2 indicating an interest in purchasing non-live data, search window 180 is displayed as set forth in FIG. 8, which may be preceded by a request for login credentials to identify the one or more acquirers. From search window 180, a data acquirer can select the one or more data types for acquisition. Note that FIG. 8 displays only a sample of potential search parameters that the system may provide, all of which are tunable parameters. Parameters can be populated initially based upon the collected data by the monetization system, which can include information provided by the user in FIG. 3A, information provided by the one or more sensors, information uploaded by the user, information derived from any of the collected information, and the like. While the system may render initial data types for acquisition, a data acquirer may have the ability to add one or more data types. Characteristically, more than one data type can be chosen at the same time for search, enabling a data acquirer to acquire multiple types of data from each individual user. After selecting the one or more data types, a data acquirer can add or select the one or more parameters related to the profile for the one or more individual(s) the acquirer is interested in acquiring data from. Each search may be done based on an acquirer’s preference for anonymized data or identifiable data (e.g., data that can be associated with a specific person or group). By clicking on identifiable data, the acquirer may be able to select all collected data from any selected user, or search data sets within any user profile or group. As an example, this may be advantageous for an insurance company that may be interested in collecting all sensor data on a specific individual or group of individuals (e.g., a specific family, a soccer team, a control group with a specific disease). In a refinement, an acquirer may be able to access both anonymized and user identifiable search results within the same search. For example, a user that may want to see anonymized data for any given parameters may have the ability to then see what identifiable individuals may be included in that search via element 184. In another refinement, animal data collected by the system is included as one or more profile search parameters for the one or more targeted individuals. For example, an acquirer may want to acquire n number of ECG data sets from individuals that have exceeded a maximum heart rate of 180 beats per minute while doing any given activity (e.g., yoga) for any given period of time (e.g., minutes). For such cases, the system can be operable to allow a data acquirer to add one or more fields that enable one or more animal data-related search parameters to be selected.

Each parameter selected in FIG. 8 results in a tag being created, which enables the monetization system to determine and locate the one or more individuals or data sets that match a given search criteria, as well as the type of data an acquirer desires (e.g., simulated data). As each individual tag is created, the system may render the number of results of the search criteria, which may include the number of users that match the criteria as well as the number of data sets available. After an initial quantity of search results are provided, the search can be narrowed and the data can be further filtered, with additional tags being created and more defined search results being rendered. For example, the monetization platform can be further programmed to search for, and identify, individuals that have collected data sets featuring one or more specific characteristics (e.g., activity, sensor used) within a desired pool of individuals. Characteristically, at least a portion of the data selected may be simulated data. A data acquirer may select simulated data for any number of reasons including cost (e.g., simulated data may be cheaper), quantity (e.g., an acquirer may be able to get more data sets of simulated data), acquisition time (e.g., it may be faster to acquire simulated data sets than real data sets), and the like. Control element 181 labeled “next” is actuated after the search criteria have been specified and the system meets the requirements of the data acquirer. In a refinement, an option to purchase artificial animal data generated by a machine may be offered to an acquirer. For example, an acquirer may want to acquire computer vision data to train artificial intelligence models for autonomous driving.

In some cases, the data acquirer performs a search based on users assigning themselves to one or more groups. A group may have a particular value based on the value provided by the group (e.g., a group that has impeccable data collection methods, and therefore a purchaser only wants to purchase data from people associated with that group) or characteristics of that group (e.g., a group with a specific medical condition, a group that is comprised of a team, a group featuring people taller than a certain height, a fitness class led by a specific instructor). In a refinement, a group can be created to signify that the data from multiple users is consistent and/or similar in one or more ways (e.g., the data was captured at the same time and in the same place and under the same conditions). Groupings may also be created by the monetization system dynamically based on one or more characteristics of the sensor data or the metadata associated with the data (e.g. the metadata may indicate that all the data was collected as part of a basketball game, or as part of a group yoga class, or as part of a data collection sleep study). Groupings or other tags may also have one or more premium values assigned by the system to the one or more data sets. In a further refinement, the monetization system may have a feedback mechanism that rates each user that provides data for a number of criteria including but not limited to collection process, willingness to provide video or images of the data collection period, willingness and degree of following directions, willingness to participate in a video-led research session, and the like.

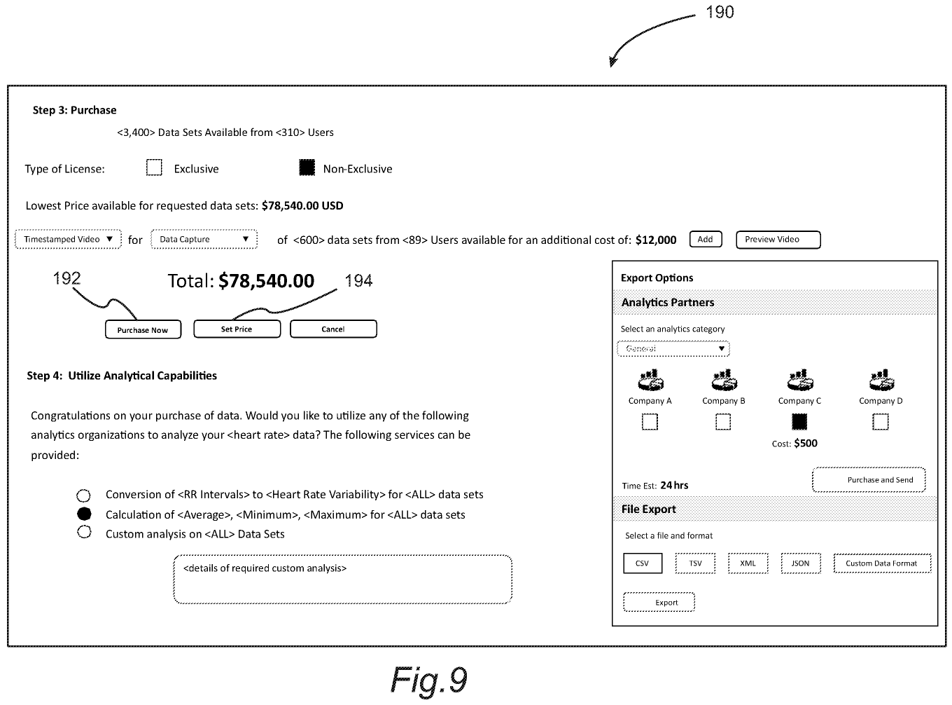

FIG. 9 provides an illustration of a purchase window 190 that is displayed after a data acquirer has found and selected the one or more data sets derived from profiles of the one or more individuals, they are interested in. A price or value proposition is created by the system based on one or more factors including the number of requested data sets, the price or associated cost each data provider charges for their data sets, terms associated with the acquisition (e.g., exclusive vs non-exclusive), and/or the premium placed upon the one or more data sets by the system. Note that one or more additional factors may be included within FIG. 9 to more finely tune the acquisition cost. This can include terms of use (e.g., type of license, along with how the data can be used, when the data can be used, where the data can be used), elements related to the contractual terms (e.g. intellectual property rights associated with the data), and the like. In the event there are multiple data providers that are in a position to provide the requested one or more data sets, the monetization system may surface the best option based on one or more data acquirer preferences (e.g., highlighting the least expensive option for the data acquirer). In a refinement, the monetization system may offer ancillary products, services, or other value offerings as part of the transaction. For example, the monetization system may offer the ability to purchase or acquire timestamped video of the data collection period in addition to the data acquired, so that an acquirer can watch the user during the period when the data was collected. In another refinement, the system may offer the acquirer an ability to preview the video and/or apply one or more artificial intelligence or machine learning techniques to determine video quality (e.g., acceptable video vs not) and usability for an acquirer (e.g., a data acquirer may want the data provider to forward face the camera at all times, and artificial intelligence techniques may enable the monetization program to identify videos that conform to this requirement vs not). In a variation, the monetization may apply one or more techniques to enhance or add value to a video, thereby creating an upsell opportunity for the monetization system. In another refinement, the acquirer may have the ability to select one or more parameters within the system to define video quality and/or usability. Once a purchase occurs via actuation of control element 192, the monetization system may provide one or more upsell opportunities (e.g., have analysis or other analytics tools applied to the purchased data). The one or more upsell opportunities (e.g., analytics tools) may be housed within the system, which may be created internally or by a third party, or sent to another system (e.g., third party analytics company). One or more of the processes related to upselling, taking one or more additional steps based upon the upsell (e.g., analyzing the data within the system), and/or sending data to another destination as part of the upsell if required (e.g., the analytics company) and retrieving it back in order to be distributed to a data acquirer can occur within the monetization platform.

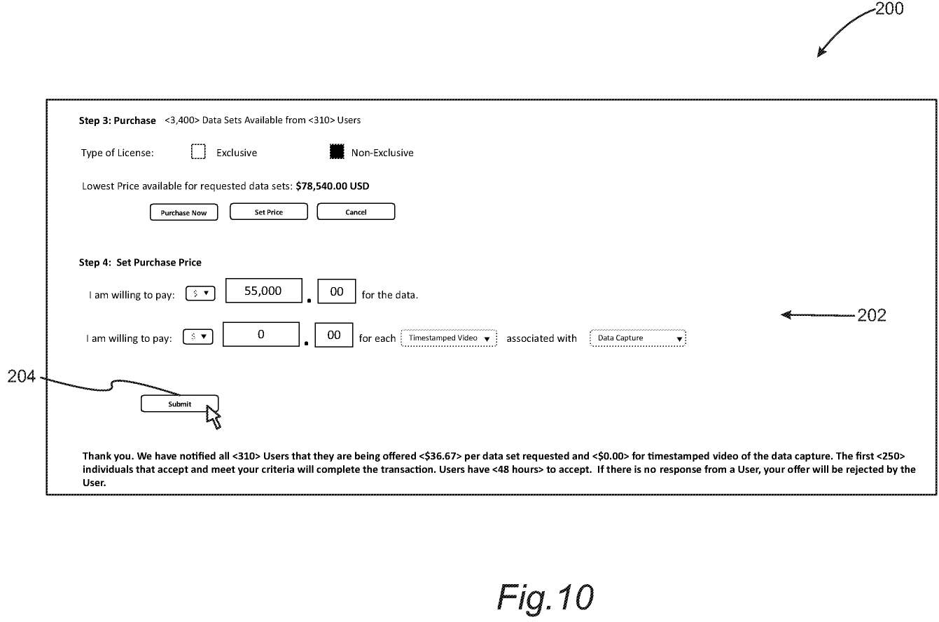

FIG. 10 provides an illustration in which window 200 includes a section 202 enabling the data acquirer to set a price for data sets and additional data-related offerings. In this scenario, a data acquirer actuates control element 194 labeled “Set Price” in FIG. 9, upon which an acquirer can set a purchase price for the data set they request (e.g., the collection of data requested). An acquirer can also set a purchase price for ancillary services or add-ons related to the data set such as timestamped video of the data capture as depicted in FIG. 10. Upon the data acquirer selecting control element 204, the monetization system will determine what the cost would be per data set (inclusive of any ancillary services if requested) and notify the data providers of the price being offered for their data. Data providers will have a specified period of time (e.g., n hours or days) to either accept or reject the offer. The specified period of time is a tunable parameter set by the acquirer or the system, and acceptance or rejection of an offer may occur within the system or via a third-party system (e.g., email application, mobile platform) that then communicates with the monetization system. The system may have a customizable default setting for data providers that do not reply or communicate with the monetization system either directly or indirectly (e.g., the offer may be automatically accepted or rejected) or data providers that want a minimum price for their data (e.g., so long as the acquisition offer is equal to or greater than the minimum price set by the data provider, the monetization system will automatically accept the offer). The system may also choose to reject an offer based upon the premium the system would retain for the requested data set (e.g., the premium the system would retain as part of a data set may be too low for the system to accept).

In a refinement, a data acquirer may desire completely new data sets from individuals with specific characteristics and desire for those individuals to follow specific instructions (e.g., when to collect, how to collect the data and what activities to do). In order to find those individuals, the data acquirer may place an “ad” with the specific characteristics, requirements & instructions and fees that will be paid to the data acquirer within the monetization system. When the data acquirer has selected the specific characteristics of the individuals, the monetization system will display the number of individuals within the monetization system that are a match. These matched individuals will be notified and given an opportunity to accept the data acquirer’s offer. An example where this type of mechanism would be useful is for sensor companies that are wanting to collect data on their sensors and increase their sample size for testing and tuning their sensor hardware, algorithms and software.

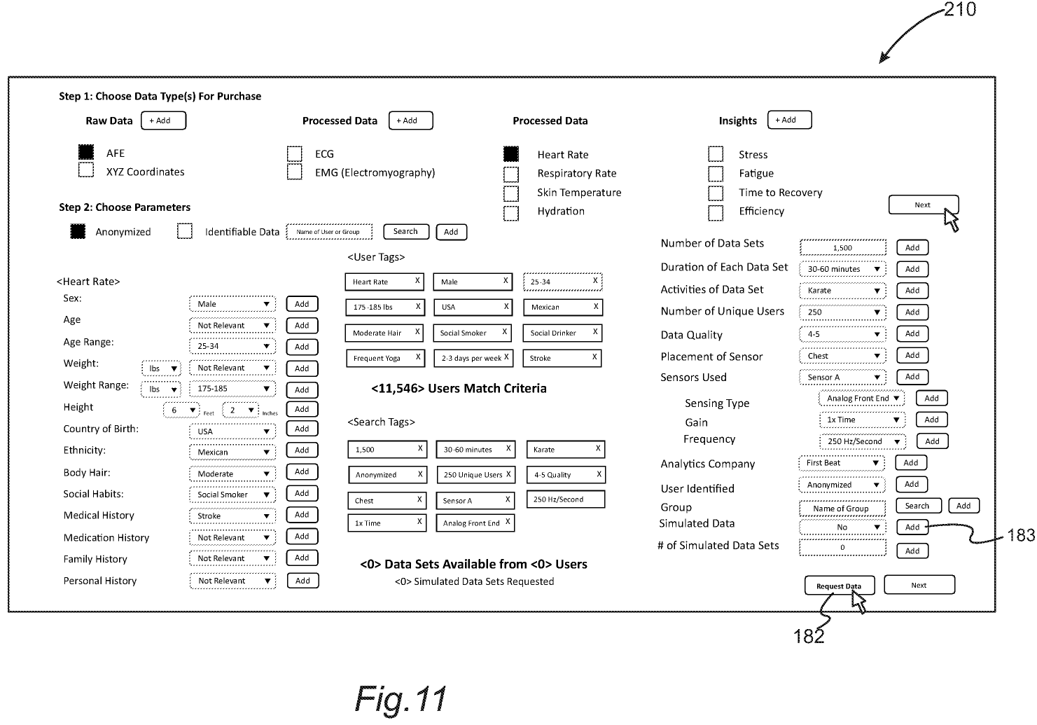

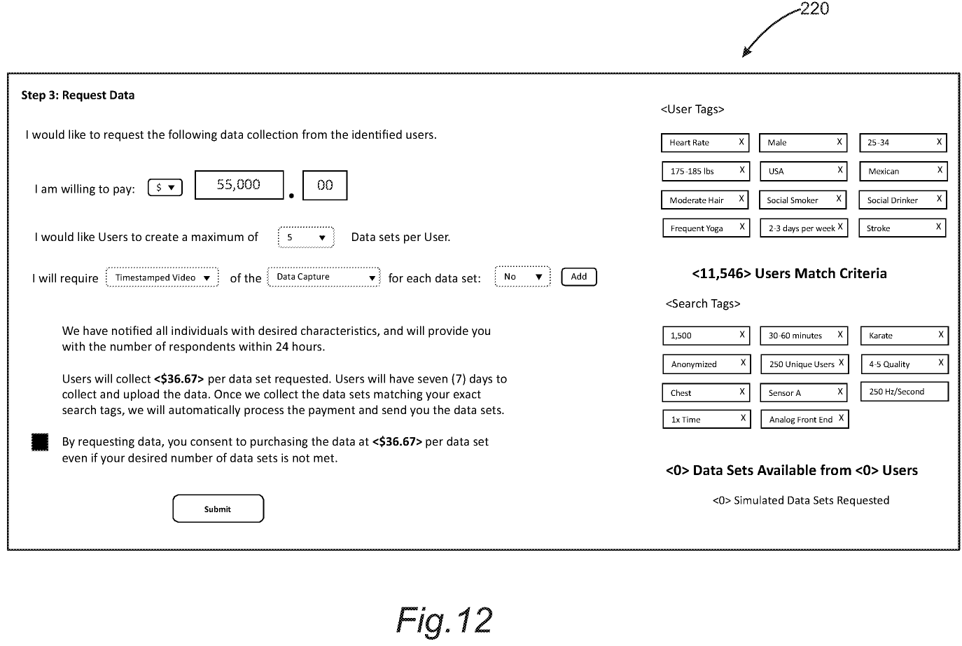

FIGS. 11 and 12 illustrate an example of a web page or window display when one or more desired data sets are selected, but the requested one or more data sets are not initially available. For example, as depicted in FIG. 11, a potential acquirer (e.g., purchaser) may search for data sets using search window 210 and find that the data sets that meet the search criteria are not available, or not available in the quantity the purchaser is looking for. Note that the user has the ability to select and add simulated data, including the number of requested simulated data sets via actuation element 183, as part of its search, which will enable the system to create one or more artificial data sets to fulfill any given request. In a refinement, the user will have the ability to select any combination of simulated data and collected user data, if available, for acquisition by a data acquirer. In another refinement, the value of the simulated data may be adjusted based on one or more variables (e.g., amount of the data utilized, data quality). For example, a larger quantity of data or more precise and accurate data used to train the one or more neural networks in a simulation may increase the value of the generated simulated data. In the event the number of data sets or number of users is less than what is required by a data acquirer’s search, and the data acquirer does not want to fulfill the request with simulated data, control element 182 labeled “request data” is actuated after the search criteria have been specified and the window depicted in FIG. 12 is displayed. In the event there are no data sets that are readily available or less than the desired number of data sets, the one or more individuals that match the one or more parameters requested by the data acquirer are contacted to determine if they are able to collect data in a manner that matches the requested one or more parameters in exchange for a fee (e.g., fee per data set or fee for all data sets collected). In a refinement, the monetization system will acquire data from one or more third parties, work with one or more analytics companies to create the data requested if they are able to derive it from collected data, create one or more analytics tools internally to derive requested data from collected data, and/or create artificial data to fulfill one or more requests for one or more data sets by a data acquirer.

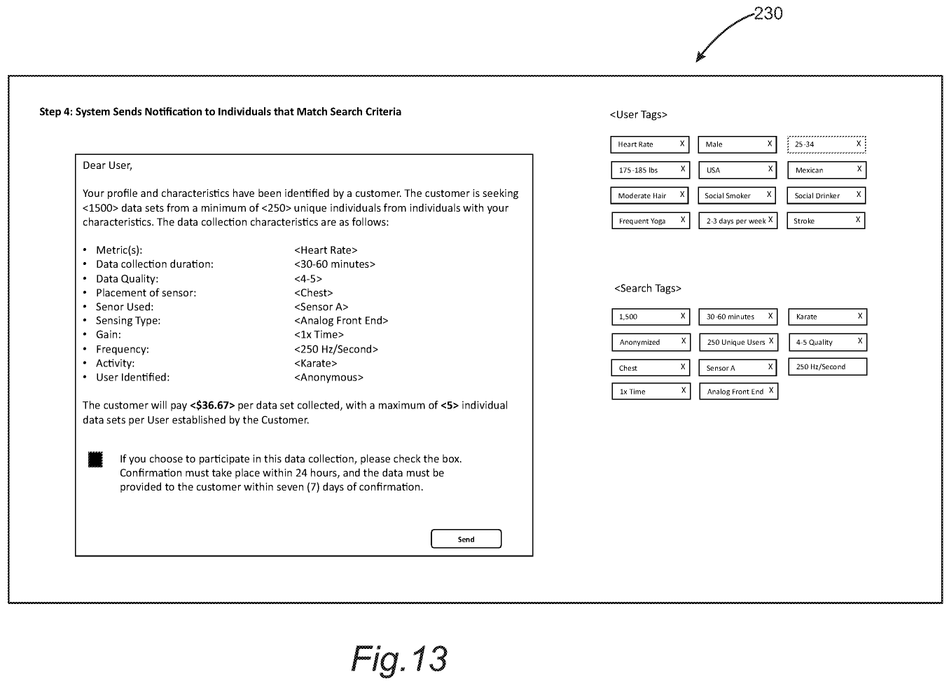

FIG. 13 provides an example of a display window 230 that a data provider would see that notifies them of the opportunity to create data to the exact specifications and parameters of the data acquirer and receive consideration for it.

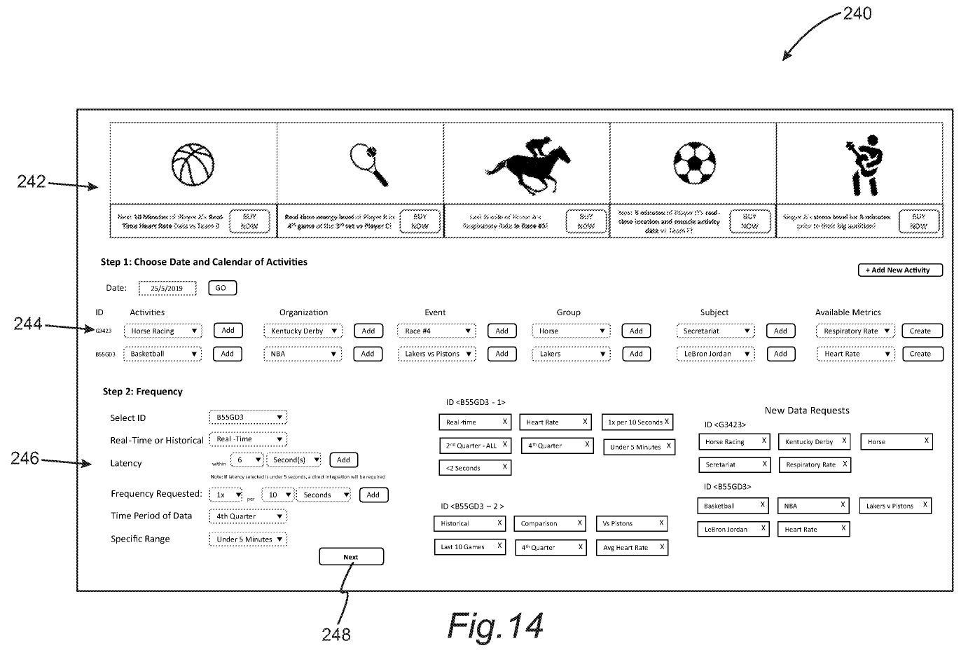

FIG. 14 illustrates the scenario when a data acquirer requests live data. To select live data, the data acquirer activates control element 104 and selection box 108 labeled “Live Data” in window 100 in FIG. 2. Upon providing login credentials to identify the data acquirer, window 240 of FIG. 14 appears showing additional information about data sets. First, at the top of the screen are trending product buys 242 that the platform could offer. For example, in the context of sports betting, such trending buys can be “Buy the Next 10 minutes of player A’s heart rate” or “Buy the last 0.5 miles of Horse A’s respiratory rate in Race #3.” In a refinement, the one or more offerings could be sent by the monetization system to a third party for display (e.g., within a sports betting platform or game-based system). If an acquirer is looking for customized data or one or more specific types of data, the acquirer can select one or more parameters (e.g., date) and see what activities are available as in customization section 244. The user will then be able to narrow down the search to obtain data that is very specific (e.g., a particular athlete’s real-time heart rate data for the last 5 minutes of the 4^{th} quarter) or very broad (e.g., a particular athlete’s real-time heart rate data for the entire season), In a refinement, the monetization system can be configured to enable more granular data searches. For example, a data acquirer may want to purchase an alert for every instance that a subject’s heart rate exceeds n beats per minute (e.g., 190 bpm) in a given match, or wants an alert when a subject’s average heart rate for any given quarter exceeds n beats per minute (e.g., 190 bpm), or wants to acquire data related to Team n’s average “energy level” in the 4^{th} quarter of the last 3 games against Team y. Note that FIG. 14 displays only a sample of potential search parameters that the system may provide (all of which are tunable parameters), and also provides an acquirer to access historical and other non-live data, A data acquirer can define their required parameters for their use case as depicted in section 246. These tunable parameters (e.g., data usage, frequency of data sent to the acquirer, and the like) can impact the cost to the acquirer.

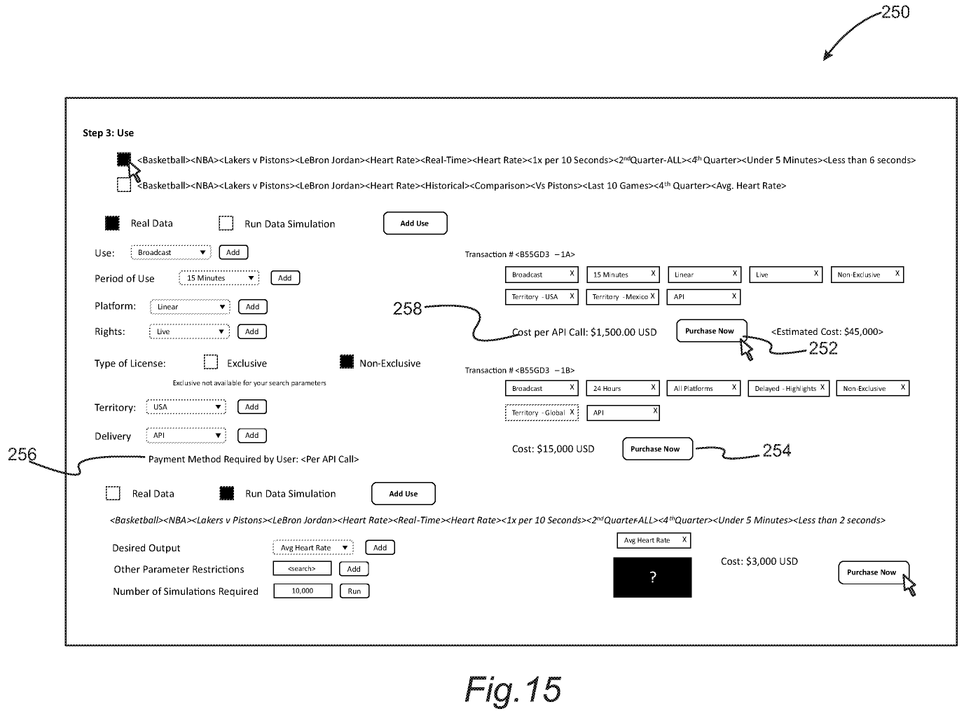

Upon defining the parameters in FIG. 14, a data acquirer actuates control element 248 labeled “next” to display window 250 in FIG. 15. FIG. 15 provides a window 250 showing one or more rights options associated with a potential acquisition (e.g., purchase). For example, if a purchaser wants heart rate data for a reality show contestant, a data purchaser may have the ability to define the rights associated with their acquisition (e.g., license), including defining territories, period of use, where the purchased data can be used (e.g., linear TV vs digital), and the like. Note that FIG. 15 displays only a sample of potential parameters that the system may provide, all of which are tunable parameters. Advantageously, the consideration model can be customized, For example, if an acquirer chooses a specific delivery method (e.g., API as in section 256), the user or administrator may have the ability to customize how the consideration is dispersed to the stakeholder(s). For example, a fee may be paid per API call as shown in section 258 or per data transfer rather than a flat acquisition fee. In this example, in the event an acquirer wants real-time heart rate data for a person over a 1 period that requires an API call once per second, the monetization system would facilitate 600 API calls and charge the acquirer for each call. The purchaser may also have the ability to run one or more data simulations and purchase the simulated data output. In any given scenario, this may be useful, for example, if a purchaser is interested in forecasting the likelihood of an outcome, or if the purchaser is interested in having the system generate a prediction. For example, if a purchaser is interested in understanding the probability that a basketball player’s heart rate will go above 190 beats per minute in the 4^{th} quarter of Game X, one or more simulations can be purchased, and occur, to create the simulated data in order to provide the desired probability output. In a refinement, the simulation system and associated fields can be configured to utilize at least a portion of animal data, simulated data, or a combination thereof, to examine one or more potential outcomes. For example, if a purchaser is interested in understanding the probability or the likelihood that Player B will win the match vs Player C utilizing at least a portion of their animal data (e.g., real-time heart rate, respiration rate, location data, biomechanical data, and the like), one or more simulations may be run to create simulated data (e.g., predicting what Player B’s animal data will look like during the match vs Player C), which can then be used in one or more further simulations to produce the desired output made available for purchase (i.e., the likelihood Player B will win the match). In another example, if an insurance company wants to know the likelihood of any given subject with specific characteristics having a medical condition (e.g., heart attack) within a defined period of time (e.g., in the next 6 months), the simulation system can identify individuals and data sets within the monetization system that share one or more characteristics with the individual (e.g., age, height, personal history, social habits, blood type, medical history, prescription history, ECG data history, heart rate history, blood pressure history, genomic/genetic history, biological fluid-derived data history) and run one or more simulations in order to determine the desired outcome made available for purchase. Note that the system can be operable to run any number of simulations across any number of subjects. Once a purchaser determines their requirements, the cost is displayed in window 250 along with control elements 252, 254 labeled “Purchase Now” to complete the purchase. In a refinement, the acquisition cost for any simulated data may be adjusted (e.g., increased) dynamically based on the one or more neural networks being provided with an opportunity to produce a more accurate output (e.g., trained with better data or higher quality data or larger quantities of more relevant data are provided). In this scenario, as the simulations get “smarter” and more accurate, the value of the data generated may increase. In another refinement, window 250 may include an ability for a data acquirer to purchase one or more simulations that utilizes at least a portion of the real animal data and/or its one or more derivatives to convert real animal data into artificial animal data for the purposes of being utilized within a video game or game based system (e.g., fitness game). In yet another refinement, the monetization system may provide an ability to acquire at least a portion of the simulated data via a third-party display (e.g., within a video game, insurance application, healthcare application).

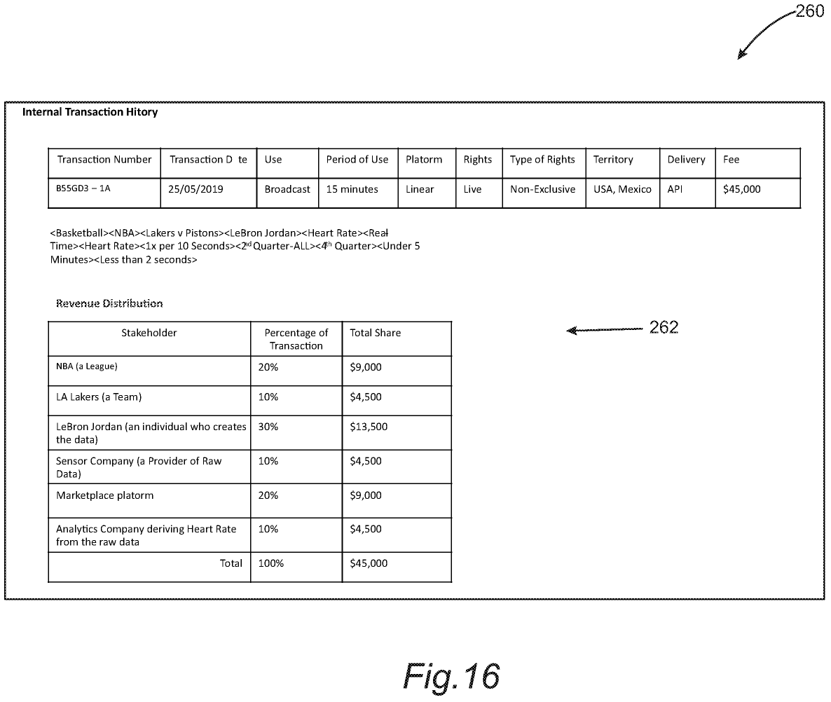

FIG. 16 provides a diagram that illustrates an example of how revenue may be dispersed from a single transaction. Record 260 illustrates that a transaction occurred and was recorded. Transaction record 262 displays the one or more stakeholders that may be part of a revenue transaction based on the value each party added. A corresponding percentage of what each stakeholder receives for contributing value to the sale of data is assigned to each stakeholder, which may change under a number of different scenarios including by transaction, by user, by data requested, and by purchaser. Percentages are a tunable parameter and may be assigned automatically by the system or manually by one or more administrators.

FIG. 17 provides a diagram of window 290 that illustrates an example of an administrator’s window for adding or removing stakeholders, and percentage of consideration sent to each stakeholder for each transaction, that may be part of any revenue transaction. The percentages are a tunable parameter, and certain use cases (e.g., live professional basketball games) may require the ability to regularly change stakeholders and percentages at any given time. In a refinement, one or more percentages are created or adjusted by one or more artificial intelligence techniques.

As depicted in FIG. 1, intermediate server 22 executes the monetization program. When implemented, the monetization program is defined by an integration layer, a transmission layer, and a data management layer. With respect to the integration layer, a user or administrator of the one or more sensors enables the monetization system to gather information from the one or more sensors in one of two ways: (1) the monetization system communicates directly with a sensor, thereby bypassing any native system that is associated with the sensor; or (2) the monetization system communicates with the cloud or native system associated with the sensor, or other system that is storing the sensor data, via an API or other mechanism to collect the data into the monetization system’s database. Direct sensor communication is achieved by either the monetization system creating new code to communicate with the sensor or the sensor manufacturer writing code to function with the monetization system. The monetization system may create a standard for communication to the monetization system that multiple sensor manufacturers may follow. The monetization system’s ability to communicate directly with the sensor may be a two-way communication, meaning the monetization system may have the ability to send one or more commands to the sensor. A command may be sent from the monetization system to the sensor to change one or more functionalities of a sensor (e.g., change the gain or sampling rate, update firmware). In some cases, a sensor may have multiple sensors within a device (e.g., accelerometer, gyroscope, ECG) which may be controlled by the monetization system. This includes the one or more sensors being turned on or off, and frequency or gain being increased or decreased. Advantageously, the monetization system’s ability to communicate directly with the one or more sensors also enables real-time or near real-time gathering of the sensor data from the sensor to the monetization system. The monetization system may have the ability to control any number of sensors, any number of functionalities, and stream any number of sensors through the single system.

The transmission layer manages direct communication with the one or more sensors or the one or more communications with the one or more clouds. With respect to direct communication with the sensor, a byproduct is that a single hardware transmission system can be utilized to (1) synchronize the communication and real-time or near real-time streaming for multiple sensors that are communicating with the monetization system directly, and (2) action upon the data itself either sending it somewhere or storing it for later use. The hardware transmission system can be configured any number of ways, can take on various form factors, can use various communication protocols (e.g., Bluetooth, ZigBee, WIFI, cellular networks), and have functionality in addition to simply transmitting data from the sensor to the system. Advantageously, the monetization system’s direct communication with the sensors enables real-time or near real-time streaming in hostile environments where potential interference or other radio frequency from other communications may be an issue.

With respect to the data management layer, the sensor data that enters the monetization system is in one of the following structures: raw (no manipulation of the data) or processed (manipulated). The monetization system may house one or more algorithms or other logic that deploy data noise filtering, data recovery techniques, and extraction or prediction techniques to extract the relevant “good” sensor data from all sensor data (both “good” and “bad”) collected, or create artificial “good” values in the event at least a portion of the sensor data is “bad.” The system may also be programmed to communicate with multiple sensors simultaneously on either a single subject or a plurality of subjects and have the ability to deduplicate them in order to transmit enough information for receiving parties to re-structure where the data is coming from and who is wearing what sensor. For clarification purposes, this means providing the system receiving the data from the system with metadata to identify characteristics of the data - for example, a given data set belongs to timestamp A, sensor B, and subject C.

Once received by the computing device, the sensor data will be sent to either the monetization system cloud or stay local on the intermediary server depending on the request made. The sensor data that enters the monetization system is synchronized and tagged by the system with information (e.g., metadata) related to the user or characteristics of the sensor including time stamps, sensor type and sensor settings, along with one or more other characteristics within the monetization system. For example, the sensor data may be assigned to a specific user. The sensor data may also be assigned to a specific event that the user is participating in (e.g., a person playing basketball in Game X), or a general class of activities that a purchaser of data would be interested in obtaining (e.g., group cycling data). The monetization system may synchronize time stamps with other non-human data sources (e.g., time stamps related to the official game clock in a basketball game, time stamps related to points scored, etc.). The monetization system, which may be schema-less and designed to ingest any type of data, will categorize the data by characteristics including data type (e.g., ECG, EMG) and data structure. The monetization system may take further action upon the sensor data once it enters the system including normalize, time stamp, aggregate, store, manipulate, denoise, enhance, organize, analyze, anonymize, synthesize, replicate, summarize, productize, and synchronize. This will ensure consistency across disparate data sets. These processes may occur in real-time or in non-real time depending on the use case and requirements of the data receiver. Given the influx of data streaming live from the one or more sensors, which may be significant in volume, the monetization system may also utilize a data management process that may include a hybrid approach of unstructured data and structured data schemas and formats. Additionally, the synchronization of all incoming data may use specific schema suitable for real-time or near real-time data transfer, reducing latency, providing error checking and a layer of security, with an ability to encrypt parts of a data packet or the entire data packet. The monetization system will communicate directly with other systems to monitor, receive, and record all requests for sensor data, and provide organizations that would like access to the sensor data with an ability to make specific requests for data that is required for their use case. For example, one request may be for 10 minutes of real-time heart rate for a specific individual at a rate of 1x per second. The monetization system will also be able to associate those requests with specific users or specific groups/classes of users.

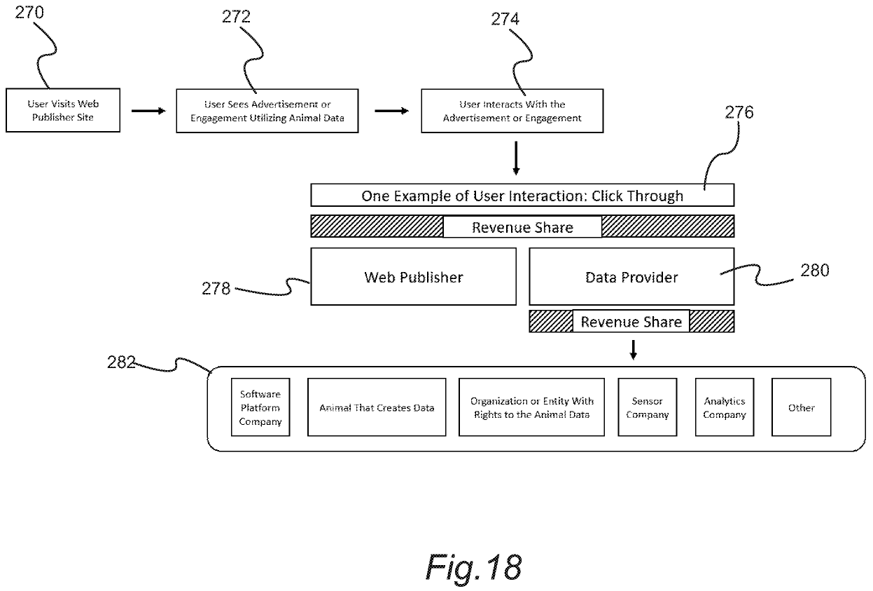

Another aspect of an effective monetization system is advertisement of the products and services provided by the system (e.g., created, offered). Animal data may be utilized, either directly or indirectly, within an advertisement, engagement, or promotion on a web page or other digital platform (e.g., within a virtual reality or augmented reality system) for the purpose of attracting a user to click through to a third-party web page or other digital destination that directly or indirectly utilizes the animal data. One way to accomplish this within a web page is by utilizing an inline frame (Iframe), which can be an HTML document embedded inside another HTML document on a website. 1 frame can be used to insert content from another source, such as an advertisement, into the web page. In some cases, the Iframe or widget is used for engagement purposes to increase a user’s time spent on a page, which can be beneficial when a page has display ads that refresh for a specified period of time (e.g., every 15 seconds), as well as to target a user to click through to another destination, which is typically a third-party site, to provide (e.g., sell) the user with a service, product, or benefit in exchange for consideration. In addition, increased time spent on a page typically leads to more highly engaged users which can lead to repeat visits to a site. There are other methods to serve in the third-party widget (e.g., JavaScript), and the present invention is not limited by these other methodologies used. FIG. 18 provides a flowchart illustrating a user interacting with a web publisher site having an advertisement for animal data (blocks 270, 272, and 274). In one specific type of advertisement, the potential data acquirer clicks through the web advertisement as indicated by block 276. Revenue from a data purchase can then be shared between the web publisher (block 278) and the stakeholders described above (blocks 280 and 282). For example, an insurance company may target one or more users within a predefined range (e.g., age, weight, height, social habits, medical history, genetic/genomic information) with a promotion to have their insurance premium lowered, an offer for an insurance quote, or an offer to obtain insurance at a specific price point if the one or more users meet a criteria defined by the insurance company based at least in part on a portion of the animal data. By the user clicking through to the third-party site to provide their animal data, the monetization system may enable the insurance company to take one or more actions (e.g., run one or more simulations to determine the probability of a person having a heart attack in the next three years based on their age, weight, height, social habits, medical history, collected animal data, and other pertinent information). In this example, based on the one or more simulations and one or more probabilities generated, the insurance company may then determine to provide the one or more users with a benefit (e.g., specified insurance rate, offer to lower a premium) based on the likelihood of one or more outcomes occurring. Upon accepting a benefit, the monetization system may enable one or more stakeholders to receive a portion of the consideration (e.g., analytics company that provided the report or ran the one or more simulations, data management company), which may be derived from the revenue generated from the new user (e.g., a portion of the premium being paid by the user) or consideration provided by the insurance company (e.g., insurance company pays monetization system for one or more services which may include data collection, running one or more simulations). In a refinement, a premium may be increased based upon at least a portion of the animal data, in which case the monetization system may receive at least a portion of the increase. In another refinement, the one or more users may request to have one or more simulations run based on at least a portion of their own animal data in order to provide information to a third party (e.g., insurance company) for the purposes of receiving a benefit (e.g., adjusting a premium or receiving another benefit). Consideration from the one or more simulations may be distributed to one or more stakeholders.

Advantageously, the products or services provided by the system may be utilized for a game-based media offering (e.g., augmented reality, virtual reality). For example, animal data may be integrated as part of an augmented reality system that enables a fan to view live sporting events with data (e.g., heart rate, “energy level”, location-based data, biomechanical data) overlaid as part of the viewing experience. A user’s consent to enable a system to use such data would enable the user and/or any other stakeholder to receive consideration in exchange for data usage. For the monetization system to provide animal data to a fan engagement system like an augmented reality system, the system may first use object recognition and tracking around a specified area (e.g., within the context of sports, around a field of play area including stadiums and fields with known boundaries and fixed objects). The system may then create an inventory of known identified scenes and tracking information along with an ability to update this information as and when required. The system may acquire known imagery data sets available to help fill in the gaps in this inventory. Using sports as an example (but not limited to sports), the AR system may use 3D tracking for the players and ancillary objects (e.g., tracking ball movement). Based on the position of the player with respect to playing field and other players, augmented objects may be placed such that the visualization is relevant to the play. Additional data from sensors like location-based data (GPS), directional sensors, accelcrometers, etc. may be used to fine tune the placement of players and bring other data points like elevation and latitude into the calculation of 3D models. The system may also look for features in the environment around the fixed known objects, and by tracking the changes in those objects with respect to some fixed point, will try to recognize and substitute relevant virtual objects in the overlay. The system will optimize data being sent to mobile devices such that rendering is in real-time or near real time. The system will use system resources either via an on-ground, aerial, or cloud-based system to render complex data sets and compute all 3D calculations. Augmented objects may include one or more types of animal data (e.g., including simulated data), or one or more derivatives from animal data, that provide information related to the one or more subjects. The augmented reality system may also include a terminal for further engagement with the data (e.g., to place a bet). The terminal and/or user’s ability to engage with the data may be controlled via a variety of mechanisms including but not limited to audio control (e.g., voice control), a physical cue (e.g., head movement, eye movement, or hand gesture), a neural cue, a control found within the AR hardware, or with a localized device (e.g., mobile phone).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A system for monetizing animal data comprising
a source of animal data that can be transmitted electronically, the source of animal data including at least one sensor; and
an intermediary server that receives and collects the animal data such that collected data has metadata attached thereto, the metadata including at least one of origination of the animal data or personal attributes of individuals from which the animal data originated, the intermediary server providing requested animal data to one or more data acquirers for consideration, the intermediary server distributing at least a portion of the consideration to at least one stakeholder, wherein the intermediary server includes a single computer server or a plurality of interacting computer servers.

2. The system of claim 1 wherein the animal data is human data.

3. The system of claim 1 wherein the animal data is assigned to one or more classifications that include metric classifications, insight classifications, personal classifications, sensor classifications, data property classifications, data timeliness classifications, or data context classifications.

4. The system of claim 3 wherein the one or more classifications associated with the animal data contribute to creating or adjusting an associated value for the animal data.

5. The system of claim 1 wherein data quality assessments of the animal data are provided as part of the metadata or separately to one or more interested parties, the data quality assessments include one or more factors selected from the group consisting of accuracy, timeliness, data consistency, and data completeness.

6. The system of claim 1 wherein the at least one sensor or its one or more appendices arc affixed to, are in contact with, or send one or more electronic communications in relation to or derived from a subject’s body, eyeball, vital organ, muscle, hair, veins, blood, biological fluid, blood vessels, tissue, or skeletal system, embedded in a targeted individual, lodged or implanted in a targeted individual, ingested by the targeted individual, integrated to comprise at least a portion of the targeted individual, or integrated as part of, or affixed to or embedded within, a fabric, textile, cloth, material, fixture, object, or apparatus that contacts or is communication with the targeted individual either directly or via one or more intermediaries.

7. The system of claim 1 wherein the sensor is a biosensor that gathers physiological, biometric, chemical, biomechanical, location, environmental, genetic, genomic, or other biological data from one or more targeted individuals.

8. The system of claim 1 wherein the at least one sensor gathers or derives at least one of facial recognition data, eye tracking data, blood flow data, blood volume data, blood pressure data, biological fluid data, body composition data, biochemical composition data, biochemical structure data, pulse data, oxygenation data, core body temperature data, skin temperature data, galvanic skin response data, perspiration data, location data, positional data, audio data, biomechanical data, hydration data, heart-based data, neurological data, genetic data, genomic data, skeletal data, muscle data, respiratory data, kinesthetic data, thoracic electrical bioimpedance data, ambient temperature data, humidity data, barometric pressure data, elevation data, or a combination thereof.

9. The system of claim 1 wherein the animal data includes one or more data sets originating from one or more sensors from one or more targeted individuals.

10. The system of claim 1 wherein a targeted individual’s data is combined with one or more data sets from one or more targeted individuals sharing at least one similar characteristic to be provided as a collection of animal data to a data acquirer.

11. The system of claim 1 wherein one or more personal attributes include at least one component selected from the group consisting of name, weight, age, height, birthdate, gender, country of origin, area of origin, race, reference identification, one or more social habits, ethnicity, one or more medical conditions, one or more locations where a targeted individual has lived, current residence, one or more activities the targeted individual is engaged in while the animal data is collected, one or more associated groups, information gathered from medical records, social habits, social data, family history, historical personal data, education records, criminal records, employment history, medication history, social media records, biological fluid-derived data, genetic-derived data, genomic-derived data, manually inputted personal data, or a combination thereof.

12. The system of claim 1 wherein the intermediary server communicates with the source of animal data either directly, through a cloud, or a local server.

13. The system of claim 1 wherein the source of animal data transmits the animal data to the intermediary server either wirelessly or utilizing a wired connection.

14. The system of claim 1 wherein the source of animal data transmits the animal data to the intermediary server with a hardware transmission system.

15. The system of claim 1 wherein the intermediary server receives the animal data in raw form or processed form.

16. The system of claim 15 wherein the intermediary server operates on the animal data by implementing one or more actions selected from the group consisting of normalizing the animal data, associating a time stamp with the animal data, aggregating the animal data, applying a tag to the animal data, storing the animal data, manipulating the animal data, denoising the animal data, enhancing the animal data, organizing the animal data, analyzing the animal data, anonymizing the animal data, visualizing the animal data, synthesizing the animal data, summarizing the animal data, synchronizing the animal data, replicating the animal data, displaying the animal data, distributing the animal data, productizing the animal data, performing bookkeeping on the animal data, and combinations thereof.

17. The system of claim 16 wherein a value is assigned to the animal data as an associated value based upon the one or more actions or adjusted based upon the one or more actions.

18. The system of claim 17 wherein the associated value is used for at least one of acquiring, buying, selling, trading, licensing, leasing advertising, rating, standardizing, certifying, researching, distributing, or brokering an acquisition, purchase, sale, trade, license, lease, or distribution of personally identified or de-identified animal data.

19. The system of claim 1 wherein the intermediary server communicates with one or more other systems to monitor, receive, and record all requests for animal data, and provide one or more data acquirers with an ability to make one or more requests for animal data by utilizing at least one of parameters that are established by the metadata, one or more search parameters, or one or more other characteristics associated with the sensor, data type, targeted individual, group of targeted individuals, or targeted output.

20. The system of claim 1 wherein upon sending the animal data to another source, the intermediary server records one or more characteristics of the animal data provided as part of a transaction, wherein the one or more characteristics of the animal data include at least one of source of the animal data, time stamps, personal attributes, type of sensor used, sensor properties, sensor parameters, sensor sampling rate, classifications, data format, type of data, algorithms used, quality of the animal data, or speed at which the animal data is provided.

21. The system of claim 1 wherein upon sending the animal data to the one or more data acquirers, the intermediary server monitors and records collection of the consideration for the animal data that was distributed.

22. The system of claim 1 wherein the animal data is offered on at least one of an eCommerce website or platform.

23. The system of claim 1 wherein a data acquirer sets a price for the animal data or places a bid for the animal data.

24. The system of claim 1 wherein a premium value on at least a portion of the animal data is placed based on one or more tags created by the system, one or more characteristics of the animal data, or one or more personal attributes of one or more targeted individuals.

25. The system of claim 1 wherein the at least one stakeholder is selected from the group consisting of a user that produced the animal data, data owner, data manager, data collection company, authorized distributor, a sensor company, an analytics company, an application company, a data visualization company, or an intermediary server company that operates the intermediary server.

26. A system for monetizing animal data comprising
a source of animal data that can be transmitted electronically, the source of animal data including at least one sensor; and
an intermediary server that receives and collects the animal data, the intermediary server providing requested animal data to one or more data acquirers for consideration, wherein at least a portion of the animal data is simulated animal data, the intermediary server distributing at least a portion of the consideration to at least one stakeholder, wherein the intermediary server includes a single computer server or a plurality of interacting computer servers.

27. The system of claim 26 wherein the simulated animal data is generated, at least in part, from collected real animal data.

28. The system of claim 26 wherein the simulated animal data is provided to a potential data acquirer with at least one parameter randomly generated.

29. The system of claim 26 wherein the simulated animal data is generated by one or more artificial intelligence techniques.

30. The system of claim 26 wherein the simulated animal data is generated from one or more trained neural networks.
